(19) United States
(12) Patent Application Publication
(10) Pub. No.: US 2023/0201383 A1
(43) Pub. Date: Jun. 29, 2023

(54) PSMA TARGETING UREA-BASED LIGANDS FOR PROSTATE CANCER RADIOTHERAPY AND IMAGING

(71) Applicants: Rigshospitalet, Copenhagen ø (DK); University of Copenhagen, Copenhagen ø (DK); Technical University of Denmark, Roskilde (DK); Deutsches Krebsforschungszentrum Stiftung Desöffentlichen Rechts, Heidelberg (DE); Albert-Ludwigs-Universitat Freiburg, Freiburg (DE)

(72) Inventors: Andreas KJÆR, Frederiksberg (DK); Matthias Manfred HERTH, Malmö (SE); Andreas Ingemann JENSEN, Virum (DK); Matthias EDER, Heidelberg (DE); Ann-Christin EDER, 79106 (DE)

(21) Appl. No.: 18/000,679

(22) PCT Filed: Jun. 4, 2021
(86) PCT No.: PCT/EP2021/065056

§ 371 Date: Dec. 5, 2022

(30) Foreign Application Priority Data
Jun. 4, 2020 (EP) 20178179.6

(51) Int. Cl. A61K 51/04

(52) U.S. Cl. CPC A61K 51/044; A61K 51/0402; A61K 51/0406; A61K 2123/00

(57) ABSTRACT

The present invention provides novel PSMA targeting urea-based ligands that binds to prostate-specific membrane antigen (PSMA) which is expressed 8-to-12-fold higher in prostate cancer cells when compared to healthy tissue. The PSMA targeting urea-based ligands comprises a chelating agent that may comprise a metal and a halogen radioisotope of fluorine, iodine, bromine or astatine. The invention further relates to a method for providing the PSMA targeting urea-based ligands of the invention, to precursors of the PSMA targeting urea-based ligands and to the PSMA targeting urea-based ligands use in radiotherapy, imaging and theranostic.

### FIELD OF THE INVENTION

The present invention relates to urea-based ligands specifically targeting a prostate-specific membrane antigen and their use in radiotherapy and imaging.

### BACKGROUND OF THE INVENTION

Prostate cancer (PC) is one of the most commonly diagnosed diseases in men.¹ Moreover, a significant amount of people suffering from PC will develop bone metastases, which results in a 1-year survival rate of only 40%. Some of the patients are non-responders to conventional hormonal therapy, developing what is known as castration-resistant prostate cancer (CRPC).^{2,3} Limited options are available for patients suffering from CRPC, for which reason the development of highly specific and potent radiopharmaceuticals is of the utmost interest. Targeting prostate-specific membrane antigen (PSMA) which is expressed 8-to-12-fold higher in PC cells when compared to healthy tissue,⁴ offers the possibility for bio-specific imaging and treatment of PC. Nevertheless, some of the developed PSMA-targeting radiopharmaceuticals have significantly unpleasant side effects, like renal toxicity and salivary gland build-up.

The use of small molecular weight ligands for selectively targeting PSMA stands to reason, as it fulfils all the requirements for radiotherapy. Small molecules exhibit the best pharmacokinetic properties such as short half-life in the bloodstream and fast clearance. In contrast, big biological entities such as antibodies have much longer circulation times and a very slow clearance profile. Long circulation time and a slow clearance profile makes a compound less suitable for radiotherapy, since the extended presence of the noxious radioactive payload in the bloodstream translates into unwanted damage in non-target tissue. In the small-molecule class, urea-based ligands have been shown to exhibit high affinities for PSMA, with very low non-specific binding, high tumour accumulation over time and fast clearance.

The current state-of-the-art in PSMA targeted radiotherapy is based on ¹⁷⁷Lu-PSMA-617, which has shown good molecular response in clinical evaluations, clearing a noticeable amount of metastases and significantly reducing PSA concentrations to normal levels (below 4.0 ng/mL).⁵ Nevertheless, a notable 30% of patients do not respond to β⁻-emitter based therapy such as ¹⁷⁷Lu-PSMA-617, for which reason an alternative strategy is to use cytotoxic alpha-emitters instead.

Efforts have been made in targeting PSMA for therapy employing alpha-emitters. For example, ²²⁵Ac has been used but it is not an ideal radionuclide for therapy as it decays through a chain that includes four α-active daughter radionuclides. Combined with a decay half-life of 10 days, this means that extensive damage can be caused to healthy tissue once the radionuclides are expelled from the chelator. Our approach is based on the use of Astatine-211 (²¹¹At) Astatine-211 is one of the most appealing radionuclides for alpha-radiotherapy. Its short half-life of 7.2 hours is in accordance with the pharmacokinetics of small-molecule urea-based PSMA ligands. Moreover, its decay pathways do not include any long-lived alpha-emitting daughter that could be expelled into the bloodstream from the binding site. This significantly reduces any unwanted cytotoxicity to the patient.

Radiohalogenated PSMA-targeting pharmaceuticals have been developed and have shown good, specific tumour uptake, but these compounds were marred by renal and salivary gland build-up, when no blocking agents were administered.⁶

Herein, we describe PSMA-targeting radiopharmaceuticals labeled with one or more nuclides or radionuclides of the halogen group, applicable for imaging, radiotherapy or theranostics, depending on the specific radionuclide or combination of radionuclides selected.

The radionuclide ²¹¹At is a therapeutic radionuclide that emits alpha particles. Alpha particles have particular properties that set them apart from other types of therapeutic radionuclides. Notably, alpha particles differ from beta particles, such as emitted by lutetium-177 (¹⁷⁷Lu), iodine-131 (¹³¹I) or yttrium-90 (⁹⁰Y), by having substantially shorter range in tissue and by depositing a higher level of energy along their path. Further, alpha particles travel by straight paths, whereas beta particles travel by tortuous paths, and alpha particle energy deposition is characterized by a Bragg peak. The shorter range of alpha particles make them more effective against micrometastases, as the energy is linearly deposited with a range of less than about 10 cancer cells. Further, the high energy deposition of alpha particles make direct double stranded DNA breaks more likely, with these having a higher chance of killing the cancer cell due to the difficulty of repair. Beta particles have less dense energy deposition, resulting in DNA damage occurring indirectly through the generation of reactive oxygen species (ROS) and being single-stranded in nature. These features make alpha particle emitters more damaging than beta emitters on a decay-by-decay basis.

Accordingly, head-to-head comparisons between alpha and beta emitters are not easily designed nor evaluated, as the two modalities have different responses in different tumor models, and they are employed at different radioactivity levels. In addition, the available relevant alpha emitters (Pb-212, Ac-225, Th-227 and At-211) also have vastly different properties, notably decay half-lives and decay chains, making each radionuclide having unique cytotoxicity and side-effect profiles.

The current state-of-the-art therapeutic variant in clinical use for beta-particle therapy is 177Lu-PSMA-617, while a variant for alpha-particle radiotherapy labeled with actinium-225 is also reported¹⁰. For theranostic imaging, gallium-68 is most commonly used. These compounds are radiolabeled in a DOTA chelator situated at the distal end of the molecule, which enables labeling with radiometals, such as Ga and Lu. However, using a chelator such as the DOTA chelator for radiolabeling is only an option in relation to radiometals and accordingly, since astatine-211 is not a radiometal but a halogen, a different strategy must be used for providing At-211 radiolabeled PSMA.

Herein, we have provided a compound series where the halogen nuclide or radionuclide, such as astatine-211, is placed in the aromatic linker region, providing a drastic difference in structure and radiolabeling technique from the compounds using radiometals. We here report that such radiochemical modifications of the linker region are possible without compromising cellular internalization, something which is considered a prerequisite for therapeutic success. Despite radioastatination of the linker region, internalization that is on par than PSMA-617 can be obtained, depending on the specific position of the astatine-211.

For compounds labeled with astatine-211 in this way, theranostic companions for imaging are highly relevant, such is analogues that are structurally identical, but with the radionuclide exchanged for e.g. fluorine-18, iodine-123, iodine-125, iodine-131 or iodine-124. These radionuclides are also halogens, like astatine-211 and are therefore well-suited for preparing theranostic companions, labeled in the same position in the linker, using related aromatic substitution radiochemistry. As per their close structural similarity, such compounds are expected to have advantages mirroring those demonstrated for the astatine-211 labeled compounds.

We have found that the compounds disclosed herein can be labeled with astatine-211 in a markedly higher radiochemical yield than reported analogous compounds, which is a substantial advantage. We believe that this may be due to the differences in molecular structure between our compounds and previously described compounds, although there is currently no reported theoretical basis for why this occurs.

Other radiopharmaceuticals developed (WO2019157037A1) were urea-based and DOTA containing, but also contained an aliphatic chain as well as a tertiary amide as key features. Tertiary amides are prone to hydrolysis in vivo^{11, 12}. The cyclohexyl group featured in our compounds has been shown to favour internalisation and thus, tumour accumulation.^{5,7} High cellular internalization is regarded as a favorable property in PSMA-targeted radiotherapy.

The PSMA targeting urea-based ligands for prostate cancer radiotherapy and imaging disclosed herein are based on peptide bonds. Thus, they do not bear any tertiary amides as the radiohalogen bearing moiety and are more stable under physiological conditions. The ease of synthesis of amide bonds through chemically modified amino acids makes these PSMA targeting urea-based ligands for prostate cancer radiotherapy and imaging easy to manufacture in an automated, resin-bound or solid-phase process if needed to scale up for routine clinical use.

The compounds disclosed herein showed excellent cellular internalization profiles. Cellular internalization data have not previously been reported for PSMA compounds modified with astatine-211 in this region of the molecule. It is therefore surprising that even with synthetic modification of the linker amino acid structure, high internalization can still be achieved, on par than optimized compounds clinically used in β⁻-particle radiotherapy (such as ¹⁷⁷Lu-PSMA-617).

### SUMMARY OF THE INVENTION

The present invention provides novel PSMA targeting urea-based ligands and the use of these compounds in radiotherapy and imaging is disclosed.

The PSMA targeting urea-based ligands of the present invention have the following general formula (I):

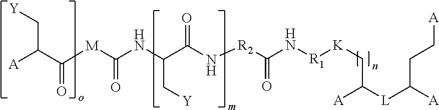

wherein:
A is independently carboxylic acid, sulphonic acid, phosponic acid, tetrazole or isoxazole;
L is selected from the group consisting of urea, thiourea, —NH—(C═O)—O—, —O—(C═O)—NH— or —CH₂—(C═O)—CH₂—,
K is selected from the group consisting of —(C═O)—NH—, —CH₂—NH—(C═O)— or

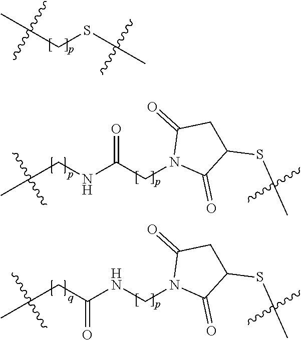

wherein
p is independently an integer selected from the group consisting of 1, 2, 3, 4, 5 and 6;
q is an integer selected from the group consisting of 0, 1, 2, 3, 4, 5 and 6;
Y is selected from the group consisting of:

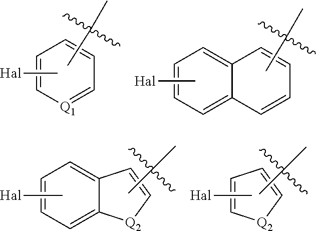

wherein
Q₁ is —C—R₃ or N, wherein R₃ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine;
M is a chelating agent, that can comprise a metal
n is an integer selected from the group consisting of 1, 2, 3, 4, 5 and 6;
m is an integer selected from the group consisting of 0 and 1;
o is an integer selected from the group consisting of 0 and 1;
R₁ is —CH—CH₂—Z or —CH—CH₂—Y;
wherein Z is selected from the group consisting of:

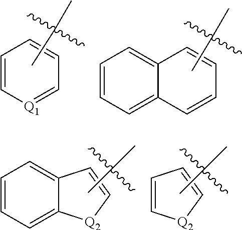

and Y is selected from the group consisting of:

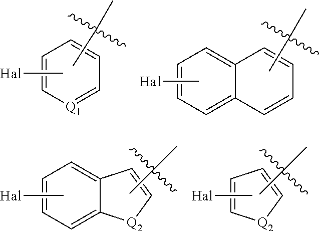

wherein
Q₁ is —C—R³ or N, wherein R³ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine;
R₂ is —CH—CH₂—Y or —CH₂—X—;
wherein X is an aromatic monocyclic or polycyclic ring system having 6 to 14 carbon atoms, cyclopropyl, cyclobutyl, cyclopentyl, cyclohexyl, cycloheptyl or cyclooctyl;
and Y is selected from the group consisting of:

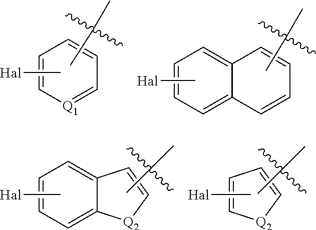

wherein
Q₁ is —C—R³ or N, wherein R³ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine;
and wherein formula (I) comprises at least one isotope or radioisotope selected from fluorine, iodine, bromine or astatine;
and pharmaceutically acceptable salts thereof.

Moreover, the invention relates to compounds of the general formula (I) but having a non-radioactive isotope of fluorine, iodine or bromine instead of a radioisotope of fluorine, iodine, bromine or astatine.

The suitability of the compounds of Formula (I) in radiotherapy and imaging is shown.

### BRIEF DESCRIPTION OF THE DRAWINGS

FIG. 1 shows the structural formula (I) and (Ia) of the compounds of the present invention.

FIG. 2A shows the time activity curve of non-target tissue for compound PSMA-617.

FIG. 2B shows the time activity curve of tumor/muscle for compound PSMA-617.

FIG. 3A shows the time activity curve of non-target tissue for compound Ii.

FIG. 3B shows the time activity curve of tumor/muscle for compound Ii.

FIG. 4A shows the time activity curve of non-target tissue for compound Ij.

FIG. 4B shows the time activity curve of tumor/muscle for compound Ij.

FIG. 5A shows the time activity curve of non-target tissue for compound Ik.

FIG. 5B shows the time activity curve of tumor/muscle for compound Ik.

FIG. 6A shows the time activity curve of non-target tissue for compound Il.

FIG. 6B shows the time activity curve of tumor/muscle for compound Il.

FIG. 7A shows the time activity curve of non-target tissue for compound Im.

FIG. 7B shows the time activity curve of tumor/muscle for compound Im.

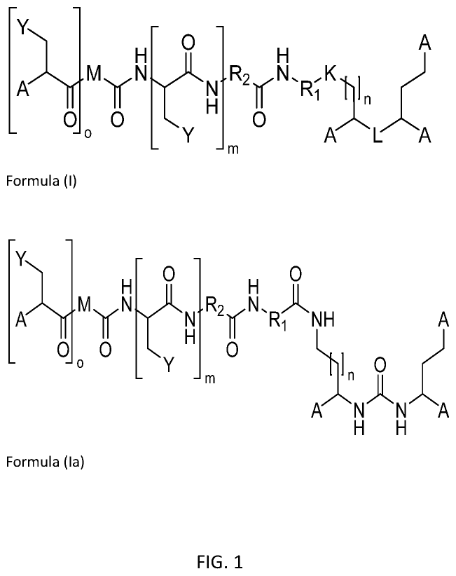

### DETAILED DESCRIPTION OF THE INVENTION

The PSMA targeting urea-based ligands of the present invention are suitable for use as radiopharmaceuticals, either as imaging agents or for the treatment of prostate cancer, or as theranostic agents.

The PSMA targeting urea-based ligands of the present invention take advantage of a urea-based binding motif (((S)-5-amino-1-carboxypentyl)carbamoyl)-L-glutamic acid). This motif specifically interacts with the PSMA antigen binding pocket. It contains an urea that forms a coordination complex with a Zn⁺² atom, which is crucial for the binding. Moreover, carboxylic acids also interact with the residues in the vicinity of the binding site, making this scaffold very convenient for PSMA-specific targeting.

The compounds disclosed herein comprises at least one isotope or radioisotope selected from the halogen group and are suitable for different purposes. The halogen astatine, particularly the radioactive radionuclide ²¹¹At, is particularly useful in alpha-particle therapy, whereas ¹⁸F and the radionuclides of iodine ¹²⁵I, ¹²³I, ¹³¹I, and ¹²⁴I, are primarily intended as theranostic companions for the astatine-211 labeled variant. In this sense, ¹⁸F and most radioisotopes of iodine are suitable for imaging. Presently, the approach is that patients be first diagnosed using diagnostic imaging, for example with a compound labeled with ¹⁸F or radioiodine, and then treated with a modality such as alpha-particle therapy. For this, analogues labeled with radionuclides for imaging are therefore required. For theranostics to work best, the diagnostic variant must have the radionuclide placed in the exact same position as the therapeutic variant, and the rest of the molecule should be identical.

The bromine radionuclides ⁷⁷Br and ⁸⁰Br are primarily relevant for Auger electron radiotherapy and ¹²⁵I and ¹²³I have also been used for such a purpose. Auger therapy is a form of radiation therapy for the treatment of cancer which relies on a large number of low-energy electrons (emitted by the Auger effect) to damage cancer cells, rather than the high-energy radiation used in traditional radiation therapy. Similar to other forms of radiation therapy, Auger therapy relies on radiation-induced damage to cancer cells (particularly DNA damage) to arrest cell division, stop tumor growth and metastasis and kill cancerous cells. It differs from other types of radiation therapy in that electrons emitted via the Auger electrons are released in large numbers with low kinetic energy.

Non-radioactive test-compounds corresponding to the radioactive compounds but comprising a non-radioactive isotope of iodine, fluorine and bromine, respectively, can be applied instead of the radioactive variants in order to test the applicability of the compounds. Such test-compounds preferably comprises one of the non-radioactive isotopes ¹²⁷I, ¹⁹F, ⁷⁹Br or ⁸¹Br, respectively, instead of the radioactive variants. There is, however, no non-radioactive isotope for astatine, but ¹²⁷I can be used as a test-compound instead. For instance, in order to test the impact of influencing the linker region, iodine ¹²⁷I isotope were provided and tested herein. ¹²⁷I is a large atom (atomic radius: 198 pm) and similar in size to astatine-211 (atomic radius: 200 pm) and with a highly similar halogen electronic configuration. Accordingly, experiments using the non-radioactive ¹²⁷I-compound was applied to demonstrate that the linker region can be modified with a large halogen and still display efficient internalization, on par with or better than reported, optimized compounds in clinical use.

Surprisingly, despite the synthetic modifications in key regions, the binding profile and biodistribution/pharmacokinetics of various of the herein disclosed new compounds are comparable or superior to values observed for PSMA-617 in direct head-to-head comparison.

This is particularly true for compounds Ii and Im. PSMA-617 is the most clinically applied therapeutic PSMA inhibitor.

The term “nuclide” comprises both non-radioactive and radioactive nuclides (radionuclides). The compounds of the present invention can comprise either a radioactive or non-radioactive nuclide depending on the intended use. When used herein in relation to specific compounds the terms “nuclide” and “radionuclide” are used to make an indication of whether the final compound is radioactive or not.

The term “isotope” comprises both non-radioactive and radioactive isotopes (radioisotopes). The compounds of the present invention can comprise either a radioactive or non-radioactive isotope depending on the intended use. When used herein in relation to specific compounds the terms “isotope” and “radioisotope” are used to make an indication of whether the final compound is radioactive or not. The various isotopes of the halogen nuclides iodine, fluorine, bromine and astatine are all well known, and the isotope number will reveal whether the isotope is stable (non-radioactive) or not (radioactive).

The PSMA targeting urea-based ligands of the present invention have the following general formula (I):

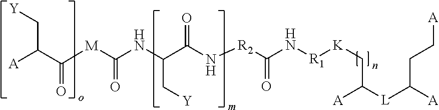

wherein:
A is independently carboxylic acid, sulphonic acid, phosponic acid, tetrazole or isoxazole;
L is selected from the group consisting of urea, thiourea, —NH—(C═O)—O—, —O—(C═O)—NH— or —CH₂—(C═O)—CH₂—;
K is selected from the group consisting of —(C═O)—NH—, —CH₂—NH—(C═O)— or

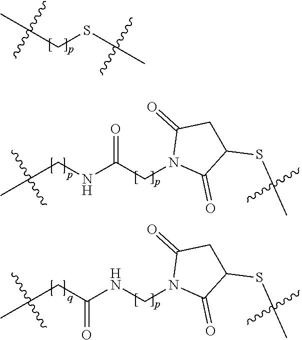

wherein
p is independently an integer selected from the group consisting of 1, 2, 3, 4, 5 and 6;
q is an integer selected from the group consisting of 0, 1, 2, 3, 4, 5 and 6;
Y is selected from the group consisting of:

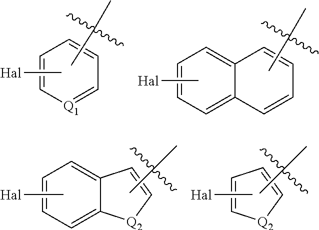

wherein
Q₁ is —C—R³ or N, wherein R³ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine; M is a chelating agent, that can comprise a metal,
n is an integer selected from the group consisting of 1, 2, 3, 4, 5 and 6;
m is an integer selected from the group consisting of 0 and 1;
o is an integer selected from the group consisting of 0 and 1;
R₁ is —CH—CH₂—Z or —CH—CH₂—Y;
wherein Z is selected from the group consisting of:

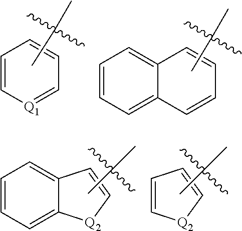

and Y is selected from the group consisting of:

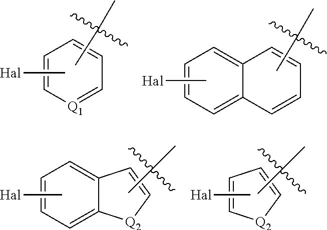

wherein
Q₁ is —C—R³ or N, wherein R³ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine;
R₂ is —CH—CH₂—Y or —CH₂—X—;
wherein X is an aromatic monocyclic or polycyclic ring system having 6 to 14 carbon atoms, cyclopropyl, cyclobutyl, cyclopentyl, cyclohexyl, cycloheptyl or cyclooctyl;
and Y is selected from the group consisting of:

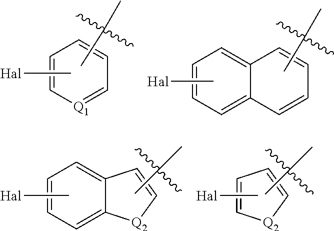

wherein
Q₁ is —C—R³ or N, wherein R³ is H or C₁-C₅ alkyl;

### Q₂ is O, S or NH;

Hal is a nuclide or radionuclide of the halogen group selected from the group consisting of isotopes and radioisotopes of fluorine, iodine, bromine or astatine;
and wherein formula (I) comprises at least one isotope or radioisotope selected from fluorine, iodine, bromine or astatine,
and pharmaceutically acceptable salts thereof.

In a particular embodiment, the PSMA targeting ligand of formula (I) is selected from the group of compounds of formula (Ia):

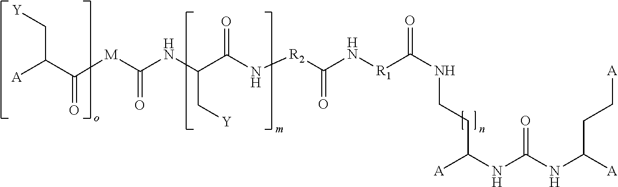

wherein:
A is independently carboxylic acid, sulphonic acid, phosponic acid, tetrazole or isoxazole;
n is an integer selected from the group consisting of 1, 2, 3 and 4;
m is an integer selected from the group consisting of 0 and 1;
o is an integer selected from the group consisting of 0 and 1;
Y is selected from the group consisting of:

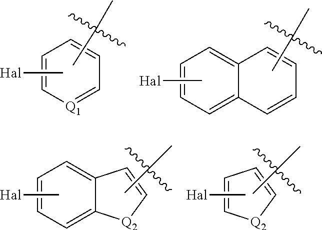

wherein
Q₁ is —C—R³ or N, wherein R³ is H or C₁-C₅ alkyl;

### Q₂ is O, S or NH;

Hal is selected from the group consisting of isotopes and radioisotopes of fluorine, iodine, bromine or astatine;
M is a chelating agent that can comprise a metal
R₁ is —CH—CH₂—Z or —CH—CH₂—Y;
wherein Z is selected from the group consisting of:

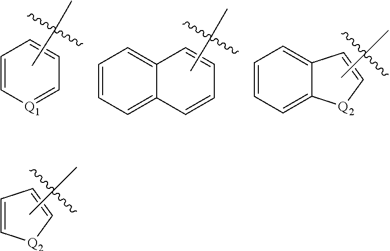

and Y is selected from the group consisting of:

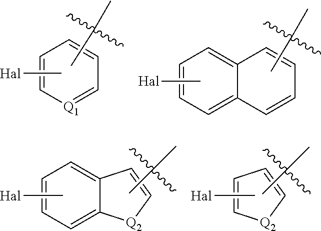

wherein
Q is —C—R³ or N, wherein R³ is H or C₁-C₅ alkyl;

### Q₂ is O, S or NH;

Hal (halogen) is selected from the group consisting of isotopes and radioisotopes of fluorine, iodine, bromine or astatine;
R₂ is —CH—CH₂—Y or —CH₂—X—;
wherein X is an aromatic monocyclic or polycyclic ring system having 6 to 14 carbon atoms, cyclopropyl, cyclobutyl, cyclopentyl, cyclohexyl, cycloheptyl or cyclooctyl;
and Y is selected from the group consisting of:

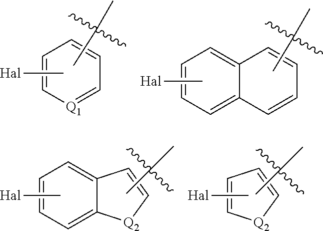

wherein
Q₁ is —C—R³ or N, wherein R³ is H or C₁-C₅ alkyl;

### Q₂ is O, S or NH;

Hal is selected from the group consisting of isotopes and radioisotopes of fluorine, iodine, bromine or astatine;
and wherein formula (I) comprises at least one isotope or radioisotope selected from fluorine, iodine, bromine or astatine;
and pharmaceutically acceptable salts thereof.

The chelating agent may be selected from one of the following chelators:
1,4,7,10-tetraazacyclododecane-N,N′,N′,N″-tetraacetic acid (DOTA),
N,N′-bis(2-hydroxy-5-(carboxyethyl)benzyl)ethylenediamine N,N′-diacetic acid (HBED-CC),
14,7-triazacyclononane-1,4,7-triacetic acid (NOTA),
2-(4,7-bis(carboxymethyl)-1,4,7-triazonan-1-yl)pentanedioic acid (NODAGA),
2-(4,7,10-tris(carboxymethyl)-1,4,7,10-tetraazacyclododecan-1-yl)pentanedioic acid (DOTAGA),
14,7-triazacyclononane phosphinic acid (TRAP),
14,7-triazacyclononane-1-methyl(2-carboxyethyl)phosphinic acid-4,7-bis(methyl(2-hydroxymethyl)phosphinic acid (NOPO),
3,6,9,15-tetraazabicyclo9.3.1.pentadeca-1(15),11,13-triene-3,6,9-triacetic acid (PCTA),
N′-(5-acetyl (hydroxy)aminopentyl-N-(5-(4-(5-aminopentyl)(hydroxy)amino-4-oxobutanoyl)amino)pentyl-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (OXO-Do3A),
p-isothiocyanatobenzyl-DTPA (SCN-BZ-DTPA),
1-(p-isothiocyanatobenzyl)-3-methyl-DTPA (1B3M),
2-(p-isothiocyanatobenzyl)-4-methyl-DTPA (1M3B),
1-(2)-methyl-4-isocyanatobenzyl-DTPA (MX-DTPA), that can comprise a metal; and pharmaceutically acceptable salts thereof.

In some embodiments, the chelating agent comprises a metal. In a preferred embodiment, the chelating agent comprises a metal selected from the group consisting of Y, Lu, Tc, Zr, In, Sm, Re, Cu, Pb, Ac, Bi, Al, Ga, Ho and Sc.

The nuclide or radionuclide (Hal) may be present in the R₁, R₂ and Y groups in Formula (I). The nuclide or radionuclide is selected from the halogen group. This group comprises isotopes and radioisotopes of Fluorine (F), Chlorine (CI), Bromine (Br), Iodine (I) and Astatine (At).

In a preferred embodiment, the halogen nuclide is a radionuclide selected from a radioisotope of fluorine, a radioisotope of iodine, a radioisotope of bromine or a radioisotope of astatine.

In another preferred embodiment, the halogen nuclide is a radionuclide selected from the group consisting of ¹⁸F, ¹²⁵I, ¹²³I, ¹³¹I, ¹²⁴I, ²¹¹At, ⁷⁷Br and ⁸⁰Br.

In a particularly preferred embodiment, the halogen nuclide is one of the following radionuclides ^{123/124/125/131}I or ²¹¹At.

In another preferred embodiment, the nuclide is non-radioactive and selected from a non-radioactive isotope of fluorine, iodine or bromine.

In a more preferred embodiment, the nuclide (Hal) is selected from the group consisting of ¹²⁷I, ¹⁹F, ⁷⁹Br and ⁸¹Br.

In preferred embodiments, the PSMA targeting ligand according to Formula (I), is one of the following seven compounds:

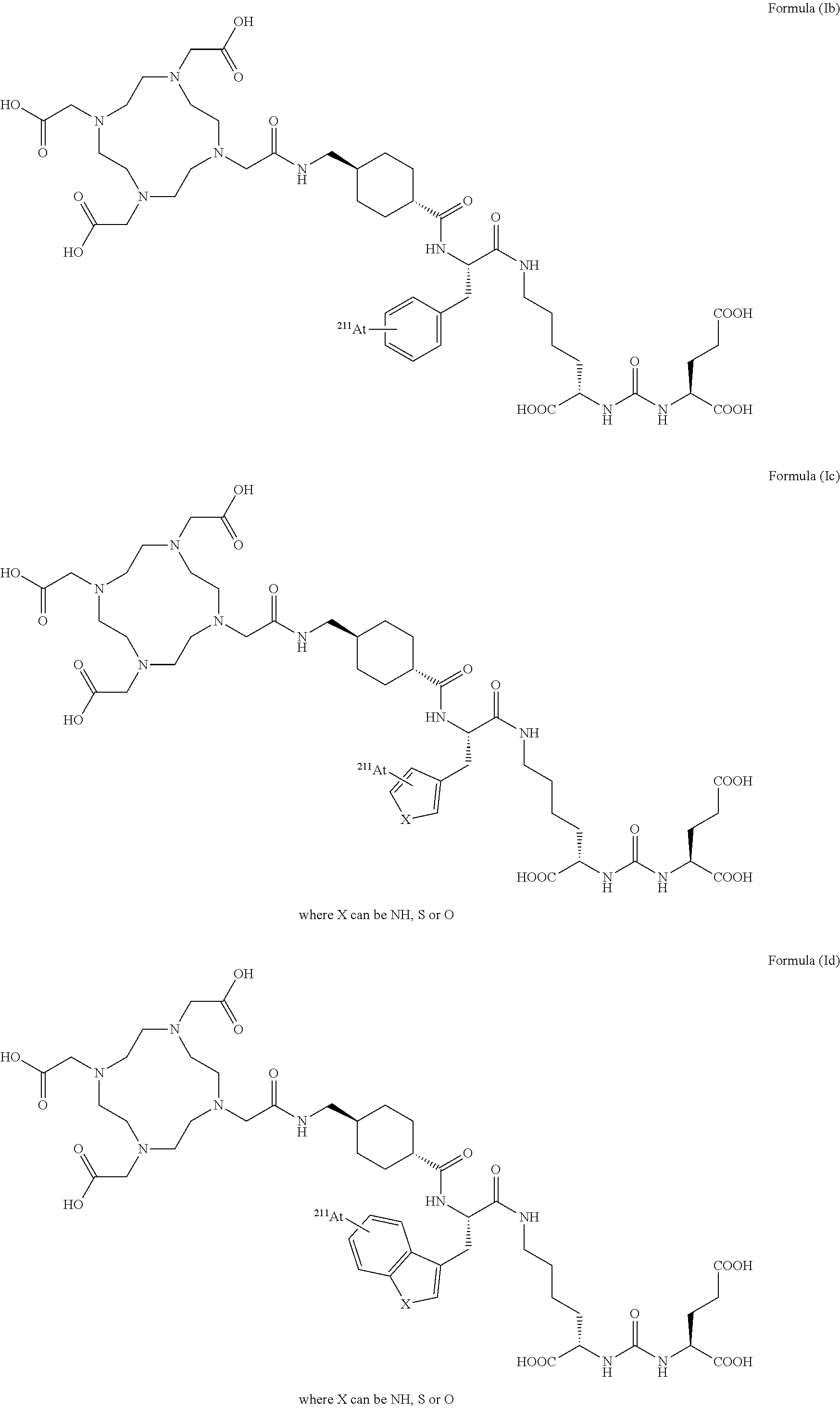

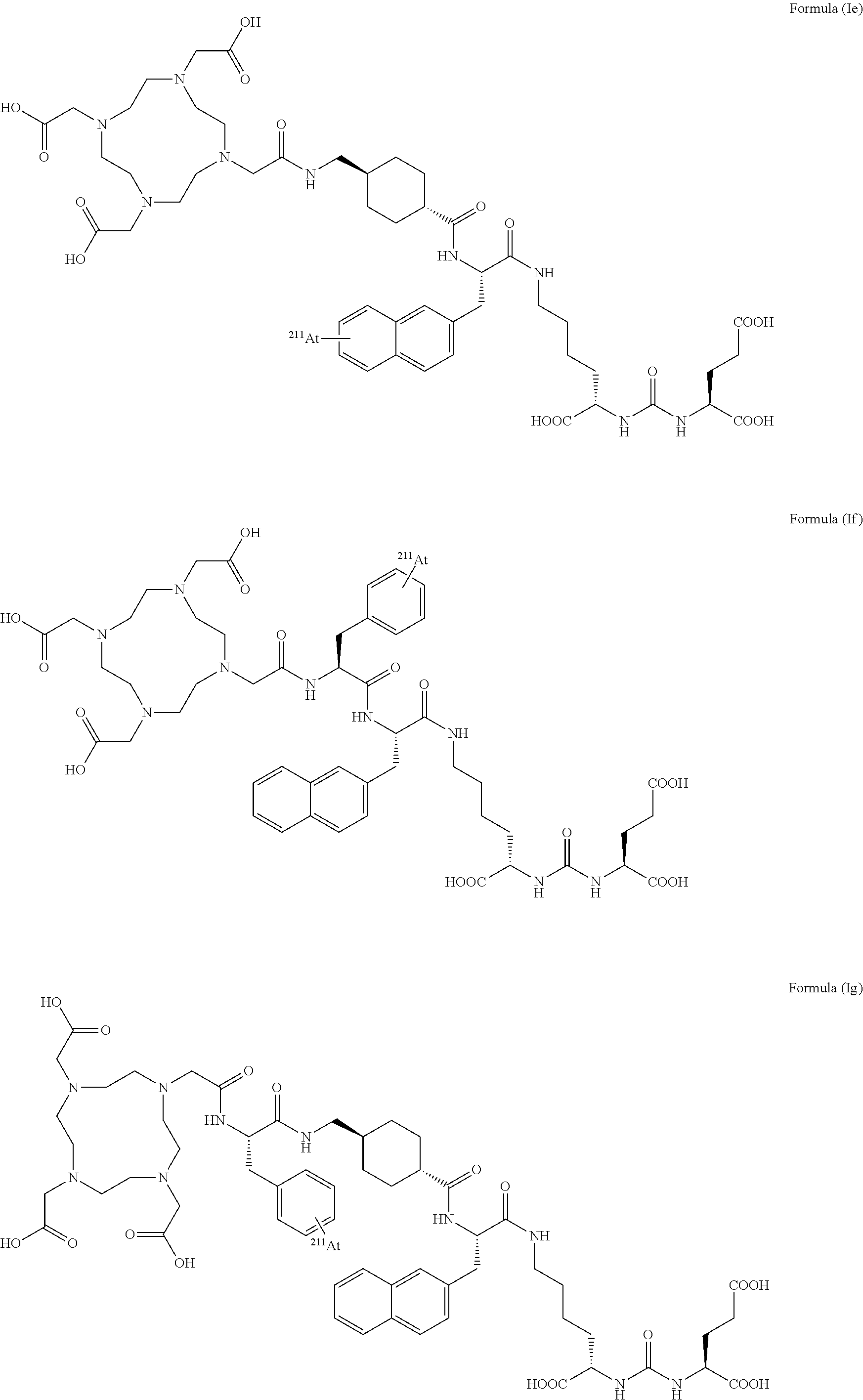

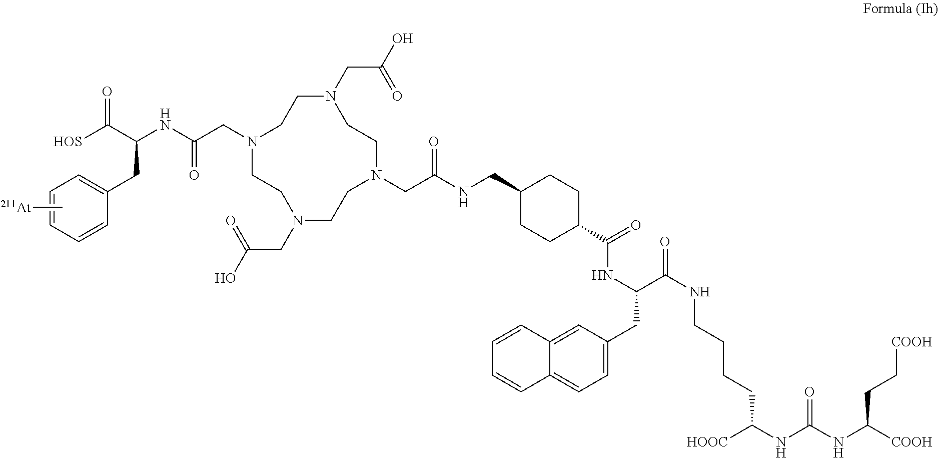

In other preferred embodiments, the PSMA targeting ligand according to Formula (I), is one of the following compounds:

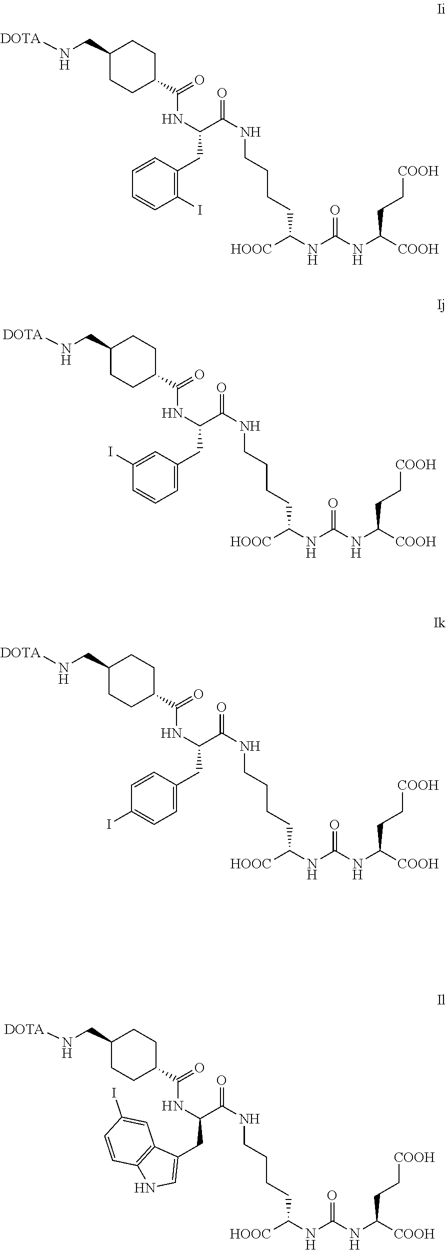

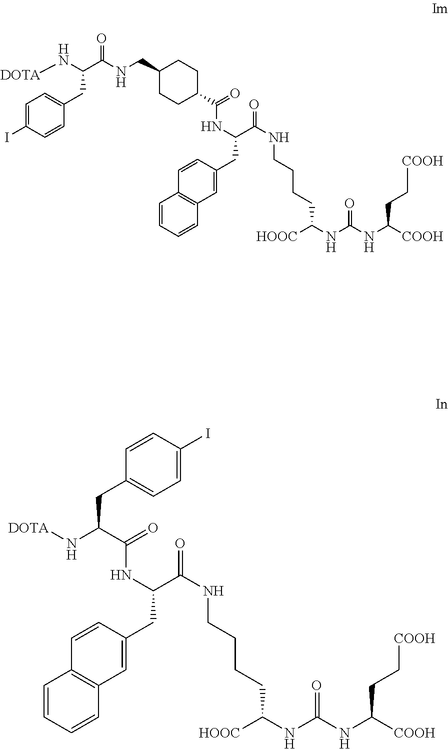

wherein iodine “I” is selected from ¹²⁷I, ¹²⁵I, ¹²³I, ¹³¹I, or ¹²⁴I.

In a most preferred embodiment, the PSMA targeting ligand according to Formula (I) is Il or Im.

The PSMA targeting ligands according to formula (I) may be provided by suitable methods known in the art.

In one aspect, the present invention relates to a method for providing the PSMA targeting ligands according to Formula (I) comprising the steps of:
Synthesis of a PSMA binding motif (BM)
Coupling of linkers to BM
Coupling of BM-linker to a chelator
Labeling the BM-linker-chelator with a halogen nuclide, such as a halogen radionuclide

In one embodiment, the PSMA binding motif is Lys-urea-Glu (LUG).

In one embodiment, the chelator is selected from:
1,4,7,10-tetraazacyclododecane-N,N′,N′,N″-tetraacetic acid (DOTA),
N,N′-bis(2-hydroxy-5-(carboxyethyl)benzyl)ethylenediamine N,N′-diacetic acid (HBED-CC),
14,7-triazacyclononane-1,4,7-triacetic acid (NOTA),
2-(4,7-bis(carboxymethyl)-1,4,7-triazonan-1-yl)pentanedioic acid (NODAGA),
2-(4,7,10-tris(carboxymethyl)-1,4,7,10-tetraazacyclododecan-1-yl)pentanedioic acid (DOTAGA),
14,7-triazacyclononane phosphinic acid (TRAP), 14,7-triazacyclononane-1-methyl(2-carboxyethyl)phosphinic acid-4,7-bis(methyl(2-hydroxymethyl)phosphinic acid (NOPO),
3,6,9,15-tetraazabicyclo9.3.1.pentadeca-1(15),11,13-triene-3,6,9-triacetic acid (PCTA),
N′-(5-acetyl (hydroxy)aminopentyl-N-(5-(4-(5-aminopentyl)(hydroxy)amino-4-oxobutanoyl)amino)pentyl-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (OXO-Do3A),
p-isothiocyanatobenzyl-DTPA (SCN-BZ-DTPA),
1-(p-isothiocyanatobenzyl)-3-methyl-DTPA (1B3M),
2-(p-isothiocyanatobenzyl)-4-methyl-DTPA (1M3B), and
1-(2)-methyl-4-isocyanatobenzyl-DTPA (MX-DTPA).

In one embodiment, the halogen radionuclide is selected from an isotope or radioisotope of fluorine, iodine, bromine or astatine.

In a preferred embodiment, the halogen is a radionuclide being one of the following radioisotopes: ¹⁸F, ¹²⁵I, ¹²³I, ¹³¹I, ¹²⁴I, ²¹¹At, ⁷⁷Br and ⁸⁰Br.

In another preferred embodiment, the halogen is a non-radioactive nuclide selected from one of the following isotopes: ¹⁹F, ¹²⁷I, ⁷⁹Br and ⁸¹Br.

The present invention also provides (Me)₃Sn precursors, silyl precursors, boron-based precursors, iodonium and diazonium salt precursors that can be used to provide the PSMA targeting ligand according to Formula (I).

These precursors include precursors with and without chelators and have the following structures, here shown for the most preferred (Me)₃Sn precursors, but the same structures are applicable for if substituting the (Me)₃Sn with silyl, boron, iodonium or diazonium:

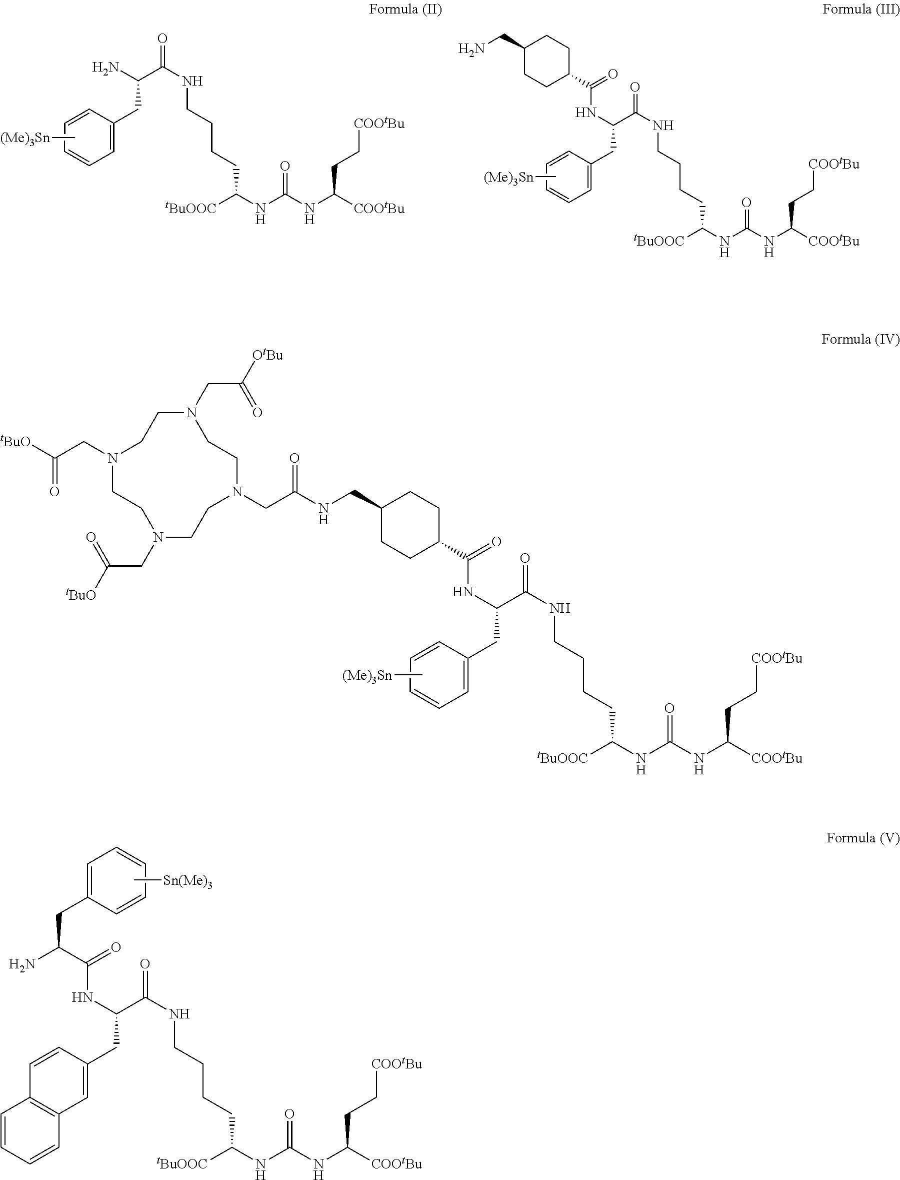

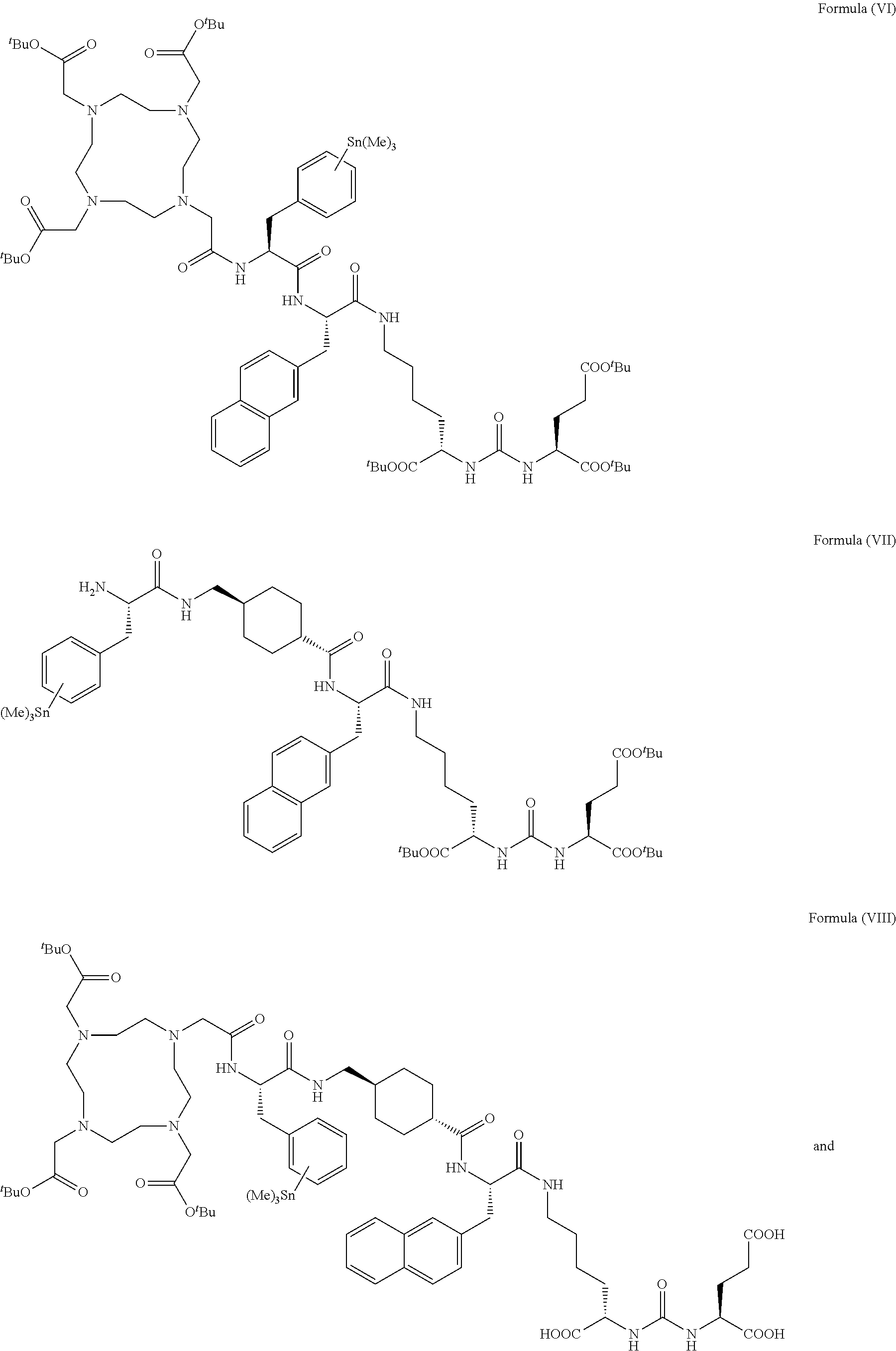

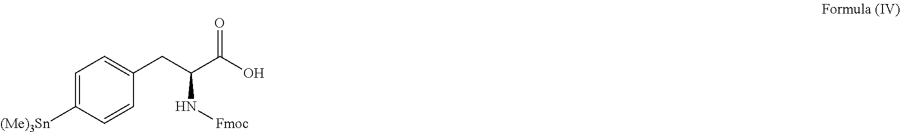

A preferred method comprises the following steps:
Synthesis of a PSMA binding motif (BM)
Coupling of linkers to BM, wherein one or more of the precursors of formula (II), (III), (V) and (VII) is provided
Coupling of BM-linker to a chelator wherein one or more of the precursors of Formula (IV), (VI) and (VIII) is provided
Labeling the BM-linker-chelator with a halogen nuclide, such as a halogen radionuclide.

In a preferred embodiment, the PSMA binding motif is Lys-urea-Glu (LUG).

The PSMA targeting ligands of formula (I) such as the PSMA targeting ligands of formula (Ia), (Ib), (Ic), (Id), (Ie), (If), (Ig), (Ih), (Ii), (Ij) (Ik), (Il), (Im) and (In) can be used in radiotherapy, as imaging agents or as both i.e. as theranostic agents.

Surprisingly, despite the synthetic modifications in key regions, the binding profile and biodistribution/pharmacokinetics of various of the herein disclosed new compounds are comparable or superior to values observed for PSMA-617 in direct head-to-head comparison. This is particularly true for compounds Ii and Im. PSMA-617 is the most clinically applied therapeutic PSMA inhibitor.

In one aspect, the PSMA targeting ligands of formula (I) are for use in radiotherapy. In a preferred embodiment the PSMA targeting ligands of formula (Ia), (Ib), (Ic), (Id), (Ie), (If), (Ig), (Ih), (Ii), (Ij), (Ik), (Il), (Im) and (In) are for use in radiotherapy. In more preferred embodiments, compounds Ii or Im are used in radiotherapy. Preferably, when using ligands of formula (I) for radiotherapy, the halogen isotope is selected from the group consisting of ²¹¹At, ¹²⁵I, ¹²³I, ⁷⁷Br, and ⁸⁰Br.

In another aspect, the PSMA targeting ligands of formula (I) are for use in the treatment of cancer, in particular prostate cancer. In a preferred embodiment, the PSMA targeting ligands of formula (Ia), (Ib), (Ic), (Id), (Ie), (If), (Ig), (Ih), (Ii), (Ij), (Ik), (Il), (Im) and (In) are used in the treatment of cancer, in particular prostate cancer. In more preferred embodiments, compounds Ii or Im are used in the treatment of cancer, in particular prostate cancer. Preferably, when using ligands of formula (I) for radiotherapy, the halogen isotope is ²¹¹At.

In yet another aspect, the PSMA targeting ligands of formula (I) are for use as a theranostic agent. In a preferred embodiment, the PSMA targeting ligands of formula (Ia), (Ib), (Ic), (Id), (Ie), (If), (Ig), (Ih), (Ii), (Ij) (Ik), (Il), (Im) and (In) are for use as theranostic agents. In more preferred embodiments, compounds Ii or Im are for use as a theranostic agents. Preferably, when using ligands of formula (I) for theranostic agents, the halogen isotope is selected from the group consisting of ¹²⁵I, ¹²³I, ¹³¹I, ¹²⁴I, ⁷⁷Br and ⁸⁰Br.

A further aspect of the invention is the use of PSMA targeting ligands of formula (I) as an imaging agent. In a preferred embodiment, the PSMA targeting ligands of formula (Ia), (Ib), (Ic), (Id), (Ie), (If), (Ig), (Ih), (Ii), (Ij), (Ik), (Il), (Im) and (In) are for use as imaging agents. In more preferred embodiments, compounds Ii or Im are for use as imaging agents. Preferably, when using ligands of formula (I) for theranostic agents, the halogen isotope is selected from the group consisting of ¹²⁵I, ¹²³I, ¹³¹I, ¹²⁴I, ⁷⁷Br and ⁸⁰Br.

A further aspect of the invention is the use of PSMA targeting ligands of formula (I) as non-radioactive test-compounds. In a preferred embodiment, the PSMA targeting ligands of formula (Ia), (Ib), (Ic), (Id), (Ie), (If), (Ig), (Ih), (Ii), (Ij), (Ik), (Il), (Im) and (In) are for use as test-compounds. In more preferred embodiments, compounds Ii or Im are for use test-compound. Preferably, when using ligands of formula (I) for theranostic agents, the halogen isotope is selected from the group consisting of ¹²⁷I, ¹⁹F, ⁷⁹Br or ⁸¹Br.

### EXAMPLES

### Example 1: General Synthetic Procedures for the Synthesis of DOTA-Bearing PSMA-Targeting Ligands

### Glu-NCO⁸:

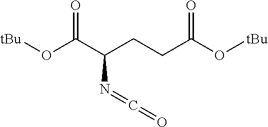 [SMILES: CC(C)(C)OC(=O)CC[C@@H](N=C=O)C(=O)OC(C)(C)C]

Firstly, di-tert-butyl L-glutamate hydrochloride (6.76 mmol) was suspended in a 1:2 mixture of dichloromethane (DCM) and saturated aqueous NaHCO₃ (72 mL). The mixture was cooled to 0° C. and then triphosgene (3.38 mmol) was added. The reaction mixture was vigorously stirred at 0° C. for 20 minutes, then warmed to room temperature, diluted with DCM and washed with brine (2×30 mL). The organic layers were collected, dried over Na₂SO₄ and concentrated in vacuo, affording the isocyanate Glu-NCO.

### PSMA Binding Motive⁸:

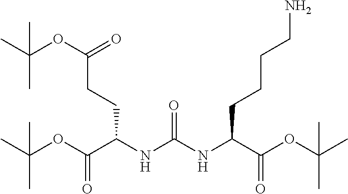 [SMILES: CC(C)(C)OC(=O)CC[C@H](NC(=O)N[C@@H](CCCCN)C(=O)OC(C)(C)C)C(=O)OC(C)(C)C]

A solution of Glu-NCO (6.73 mmol) in DCM (16 mL) was added to a mixture of tert-butyl N⁶-((benzyloxy)carbonyl)-L-lysinate hydrochloride (7.40 mmol) and dry pyridine (7.40 mmol) in DCM (50 mL). The reaction was stirred at room temperature 18 hours. Afterwards, the reaction was diluted with 10 mL of DCM, washed with 0.1M HCl (5×15 mL) and washed with brine (2×15 mL). The organic layers were collected, dried over sodium sulphate and evaporated in vacuo. Finally, the obtained crude was purified by flash chromatography.

The Cbz-protected product (3.01 mmol) was mixed with Pd/C (10%) (0.31 mmol) catalyst, dissolved in MeOH (15 mL) and hydrogen gas was bubbled through the mixture overnight. The reaction mixture was filtered through a pad of diatomaceous earth and the solvent was evaporated in vacuo, yielding the PSMA binding motive.

### PSMA Binding Motive-Linker:

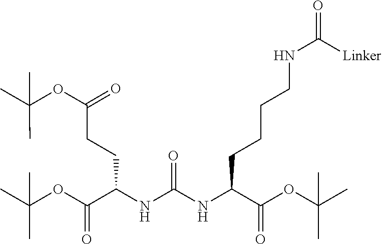

The subsequent syntheses of the PSMA-targeting peptidomimetics were carried out with the following general procedure:

N-Fmoc-amino acid (0.21 mmol) and diisopropylethylamine (DIPEA) (0.51 mmol) were dissolved in dry dimethylformamide (DMF), 1-[Bis(dimethylamino)methylene]-1H-1,2,3-triazolo[4,5-b]pyridinium 3-oxid hexafluorophosphate (HATU) (0.31 mmol) was added to the previous solution and stirred for 10 minutes. PSMA binding motive (0.21 mmol) was dissolved in dry DMF and dropwise added to the previous mixture for a total volume of 1 mL. The reaction mixture was stirred at room temperature for 4 to 24 hours depending on when completion was attained.

Thereafter, the N-terminus was deprotected by adding piperidine (50% relative to DMF) and stirring for 2 hours. Reaction mixture was poured over water and extracted with DCM. Organic layers were dried over Na₂SO₄ and volatiles removed in vacuo. The crude was purified by flash chromatography giving the desired free amine.

### PSMA Binding Motive-Linker-Chelator:

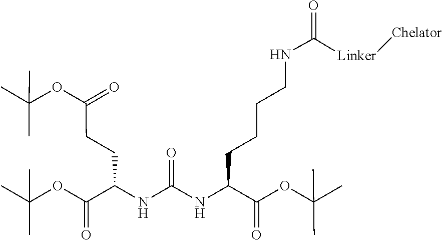

To a PSMA binding motive-linker construct solution (0.2 mmol) in either DCM or DMF (1 mL), trimethylamine (Et₃N) (0.31 mmol), DOTA-mono-NHS-tris(tBu-ester) (0.31 mmol) was added and stirred for 18 hours at room temperature. The subsequent crude mixture was purified by preparative HPLC.

### PSMA Binding Motive-Linker-Chelator Radiolabelling:

A tert-butyl protected PSMA binding motive-linker-chelator construct was dissolved in a solution of Chloramine-T, methanol, ²¹¹At and acetic acid. The mixture was stirred and reacted during 30 minutes at room temperature. Afterwards, the mixture was dried by use of a nitrogen stream. The molecule was deprotected by addition of trifluoroacetic acid (TFA) and heating to 60° C. for 30 minutes. Once fully deprotected, the mixture was dried, redissolved in a 50:50 mixture of acetonitrile (MeCN) water and purified by preparative HPLC.

### Example 2: Synthesis of PSMA-Targeting Radiopharmaceutical 1

Using the synthesis methods described in Example I, the following PSMA-targeting ligand was provided:

Synthesis of PSMA binding motif following the procedures previously described in Example I:

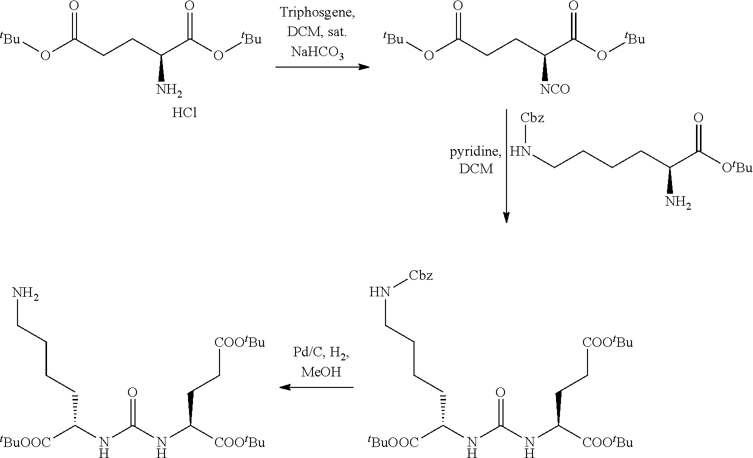

### di-tert-butyl (S)-2-isocyanatopentanedioate

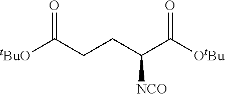

Di-tert-butyl L-glutamate hydrochloride (2.00 g, 6.76 mmol, 1.0 eq.) was suspended in DCM (24 mL) and sat. NaHCO_{3 (aq.)} (48 mL). The mixture was cooled to 0° C. and then bis(trichloromethyl) carbonate (triphosgene) (1.00 g, 3.38 mmol, 0.5 eq.) was added (OBS: triphosgene is highly toxic and must be handled with extreme care). The reaction mixture was vigorously stirred at 0° C. for 20 minutes, then allowed to warm to room temperature, diluted with DCM (36 mL) and water (30 mL) and extracted with DCM (1×25 mL). The organic layers were washed with brine (1×20 mL), dried over Na₂SO₄ and concentrated in vacuo, affording the title compound as a transparent liquid (1.92 g, 6.73 mmol, quantitative).

¹H-NMR (600 MHz, CDCl₃) δ 3.97 (dd, J=8.6, 4.5 Hz, 1H), 2.40-2.28 (m, 2H), 2.17-2.08 (m, 1H), 1.91 (m, 1H), 1.49 (d, J=0.9 Hz, 9H), 1.44 (d, J=0.9 Hz, 9H). ¹³C NMR (151 MHz, CDCl₃) δ 171.8, 170.1, 127.4, 83.7, 80.9, 57.5, 31.6, 29.2, 28.2. MS (ESI) m/z: 260.2 [M+3H—CO]⁺ (hydrolysed product)

### tri-tert-butyl (9S,13S)-3,11-dioxo-1-phenyl-2-oxa-4,10,12-triazapentadecane-9,13,15-tricarboxylate

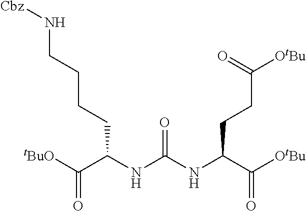 [SMILES: CC(C)(C)OC(=O)CC[C@H](NC(=O)N[C@@H](CCCCNC(=O)OCc1ccccc1)C(=O)OC(C)(C)C)C(=O)OC(C)(C)C]

A solution of di-tert-butyl (S)-2-isocyanatopentanedioate (1.92 g, 6.73 mmol, 1.0 eq.) in dry DCM (16 mL) was added to a mixture of tert-butyl N⁶-((benzyloxy)carbonyl)-L-lysinate hydrochloride (2.76 g, 7.40 mmol, 1.1 eq.) and dry pyridine (596 μL, 7.40 mmol, 1.1 eq.) in dry DCM (50 mL). The reaction was stirred at room temperature for 18 hours. Afterwards, the reaction was diluted with DCM (10 mL), washed with 0.1 M HCl_{(aq.)} (5×15 mL) and brine (15 mL). The organic fraction was collected, dried over Na₂SO₄ and evaporated in vacuo. The crude was purified by CombiFlash (heptane:EtOAc—100:0 to 20:80) to isolate the title compound as a viscous colourless oil (2.93 g, 4.71 mmol, 70%).

¹H NMR (400 MHz, CDCl₃) δ 7.37-7.28 (m, 5H), 5.22-5.05 (m, 5H), 4.36-4.29 (m, 2H), 3.22-3.12 (m, 2H), 2.35-2.22 (m, 2H), 2.09-2.02 (m, 1H), 1.87-1.67 (m, 3H), 1.66-1.23 (m, 31H) ¹³C NMR (151 MHz, CDCl₃) δ 172.6, 172.5, 171.3, 157.1, 156.8, 136.9, 128.6, 128.2, 82.2, 81.8, 80.6, 66.7, 53.4, 53.1, 40.8, 32.8, 32.0, 29.5, 29.1, 28.5, 28.2, 28.1, 22.8, 22.4. MS (ESI) m/z: 622.4 [M+H]⁺ R_{f} (40% EtOAc in heptane)=0.42

### di-tert-butyl (((S)-6-amino-1-(tert-butoxy)-1-oxohexan-2-yl)carbamoyl)-L-glutamate

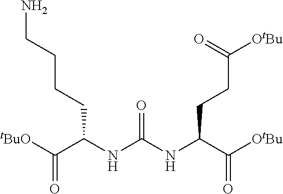 [SMILES: CC(C)(C)OC(=O)CC[C@H](NC(=O)N[C@@H](CCCCN)C(=O)OC(C)(C)C)C(=O)OC(C)(C)C]

To a flask containing tri-tert-butyl (9S,13S)-3,11-dioxo-1-phenyl-2-oxa-4,10,12-triazapentadecane-9,13,15-tricarboxylate (1.90 g, 3.06 mmol, 1.0 eq.) was added 10 wt. % Pd/C (325 mg, 0.31 mmol, 0.1 eq.) and suspended in MeOH (15 mL). The reaction vessel was purged with nitrogen and hydrogen gas was bubbled through the suspension overnight at atmospheric pressure (balloon). The reaction mixture was filtered over a pad of diatomaceous earth (Celite®) and volatiles removed in vacuo to yield di-tert-butyl (((S)-6-amino-1-(tert-butoxy)-1-oxohexan-2-yl)carbamoyl)-L-glutamate as a viscous oil (1.49 g, 3.05 mmol, 99%).

¹H NMR (600 MHz, CDCl₃) δ 5.23 (d, J=8.0 Hz, 2H), 4.32 (q, J=7.9, 4.9 Hz, 2H), 2.67 (t, J=6.8 Hz, 2H), 2.37-2.22 (m, 2H), 2.10-2.01 (m, 1H), 1.87-1.72 (m, 1H), 1.65-1.57 (m, 1H), 1.55-1.24 (m, 31H). ¹³C NMR (151 MHz, CDCl₃) δ 172.7, 172.6, 172.4, 157.0, 82.1, 81.8, 80.7, 53.6, 53.2, 41.9, 33.2, 33.0, 31.8, 28.5, 28.20, 28.16, 28.13, 22.5. MS (ESI) m/z: 488.4 [M++H]⁺

Coupling of linkers to the PSMA binding motive and labelling with ²¹¹At following the procedures previously described in Example I.

### Example 3: Synthesis of PSMA-Targeting Radiopharmaceutical Reference Compounds

### 2,5-dioxopyrrolidin-1-yl (1r,4r)-4-(((tert-butoxycarbonyl)amino)methyl)cyclohexane-1-carboxylate

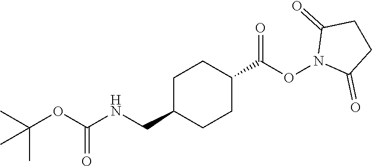 [SMILES: CC(C)(C)OC(=O)NC[C@H]1CC[C@H](C(=O)ON2C(=O)CCC2=O)CC1]

Boc-tranexamic acid (3.00 g, 11.66 mmol) was dissolved in dry THF (100 mL). Thereafter EDC-HCl (3.36 g, 17.48 mmol) and N-hydroxysuccinimide (2.00 g, 17.48 mmol) were added as solids. The mixture was stirred at room temperature and the progress followed by UPLC-MS. The reaction mixture was a cloudy suspension that gradually became a clear solution over 4 hours of stirring. After 72 hours the reaction mixture was diluted with DCM (75 mL), washed with water (3×30 mL), dried over Mg₂SO₄, filtered and solvents evaporated under reduced pressure to yield 2,5-dioxopyrrolidin-1-yl (1r,4r)-4-(((tert-butoxycarbonyl)amino)methyl)cyclohexane-1-carboxylate as a white powder (2.6 g, 7.25 mmol, 63%).

¹H NMR (400 MHz, CDCl₃) δ 2.99 (t, J=6.5 Hz, 2H), 2.82 (s, 4H), 2.58 (tt, J=12.2, 3.6 Hz, 1H), 2.17 (dd, J=13.9, 3.6 Hz, 2H), 1.88 (dd, J=13.7, 3.5 Hz, 2H), 1.60 (dd, J=13.0, 3.4 Hz, 2H), 1.55 (d, J=2.8 Hz, 1H), 1.44 (s, 9H), 1.01 (qd, J=13.2, 3.6 Hz, 2H). ¹³C NMR (101 MHz, CDCl₃) δ 170.9, 169.3, 156.2, 79.4, 46.5, 40.8, 37.6, 29.5, 28.5, 28.4, 25.7. MS (ESI) m/z: 255.3 [M+H-Boc]⁺

### General Procedure I: Fmoc Solution-Phase Synthesis of LuG-Linker Molecules

N-Fmoc-amino acid (Fmoc-AA) (1.0 eq) and DIPEA (2.5 eq) were dissolved in dry DMF (1-3 mL), HATU (1.5 eq) was added to the previous solution and stirred for 15 minutes. The corresponding amine (1.0 eq) was dissolved in dry DMF (1-3 mL) and added to the previous mixture for a total volume of 2-6 mL. The reaction mixture was stirred at room temperature, until completion (5 to 24 hours). Thereafter, the Fmoc-protected N-terminus was deprotected by adding piperidine (50% vol. relative to DMF) and stirred for an additional 2 hours. Then, the reaction mixture was poured over water (10 mL) and extracted with DCM (2×15 mL). The combined organic layers were washed with water (3×10 mL), dried over MgSO₄ and volatiles removed in vacuo. The crude was purified by CombiFlash giving the desired free amine.

### General Procedure II: Synthesis of Tranexamic Acid-Peptide Conjugates

Zwitter ionic alpha-amino acid (1.2 eq.) and Na₂CO₃ (2.0 eq.) were dissolved in 20 mL of a 1:2 mixture of H₂O/1,4-dioxane. Afterwards, a solution of 2,5-dioxopyrrolidin-1-yl (1r,4r)-4-(((tert-butoxycarbonyl)amino)methyl)cyclohexane-1-carboxylate (1.0 eq.) in 1,4-dioxane (5 mL) was added. The mixture was stirred for 4 hours, where completion was observed by UPLC-MS. Thereafter, the pH of the mixture was adjusted to 3 with 1M HCl_{(aq.)} and the precipitated product was extracted with EtOAc (3×15 mL), dried over Mg₂SO₄, filtered and solvents evaporated in vacuo to yield the desired compound as a white powder.

### General Procedure III: Synthesis of Boc-LuG-Linker Molecules

Carboxylic acid (1.0 eq) and DIPEA (2.5 eq) were dissolved in dry DMF (1-3 mL), HATU (1.5 eq) was added to the previous solution and stirred for 15 minutes. 12 (1.0 eq) was dissolved in dry DMF (1-3 mL) and added to the previous mixture for a total volume of 2-6 mL. The reaction mixture was stirred at room temperature for 5 to 24 hours depending on when completion was attained (followed by UPLC-MS). Once completed, the mixture was poured into 20 mL of water and extracted with DCM (3×15 mL). The organic fractions were washed once more with water (50 mL) to fully remove the DMF, dried over magnesium sulfate, filtered and solvents removed in vacuo. Crude was purified by CombiFlash (Heptane:EtOAc—100:0 to 0:100 with a gradual increase of 20% EtOAc every 6 minutes). Fractions containing desired product were combined and volatiles removed under reduced pressure to obtain the compounds.

### General Procedure IV: Deprotection of Boc-LUG-Linker Molecules

tu-Boc-protected molecule was dissolved in a 1:1 mixture of TFA:DCM (5 mL). The solution was stirred at room temperature for 2 hours while being monitored by UPLC-MS. Once the deprotection was complete the mixture was evaporated under reduced pressure. Thereafter, the compounds were left under high vacuum for 72 hours to fully remove TFA traces.

### General Procedures V and VI: Synthesis of DOTA-Linker-LUG Compounds

OBS: materials used to handle DOTA molecules are all free from metal, either glass or plastic, to avoid potential undesired chelation.

V—for Fmoc route: To an LuG-linker solution (1 eq.) in DCM, Et₃N (6 eq.), DOTA-mono-NHS-tris(Su-ester) (1.2 eq.) were added and stirred overnight (˜16 hours). Afterwards the reaction mixture was evaporated under reduced pressure and the resulting crude re-dissolved in 1:1 mixture of TFA/DCM (3 mL) and mechanically shaken for 3 hours. Thereafter, volatiles were removed in vacuo and the oily crude purified by preparatory HPLC. Fractions containing desired product were collected and lyophilized to obtain the compounds as white solids.

VI—for Boc route: To a LuG-linker solution (1 eq.) in DMF, Et₃N (6 eq.), DOTA-mono-NHS-ester (1.2 eq.) were added and stirred overnight (˜16 hours). Afterwards the reaction mixture was placed under a stream of air until dryness. Thereafter, the oily crude was purified by preparatory HPLC. Fractions containing desired product were collected and lyophilized to obtain the compounds as white solids.

### di-tert-butyl (((S)-6-((S)-2-amino-3-(naphthalen-2-yl)propanamido)-1-(tert-butoxy)-1-oxohexan-2-yl)carbamoyl)-L-glutamate

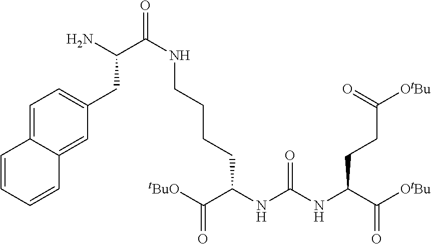 [SMILES: CC(C)(C)OC(=O)CC[C@H](NC(=O)N[C@@H](CCCCNC(=O)[C@@H](N)Cc1ccc2ccccc2c1)C(=O)OC(C)(C)C)C(=O)OC(C)(C)C]

di-tert-butyl (((S)-6-((S)-2-amino-3-(naphthalen-2-yl)propanamido)-1-(tert-butoxy)-1-oxohexan-2-yl)carbamoyl)-L-glutamate was prepared from di-tert-butyl (((S)-6-amino-1-(tert-butoxy)-1-oxohexan-2-yl)carbamoyl)-L-glutamate (500 mg, 1.02 mmol) following general procedure I employing (S)-2-((((9H-fluoren-9-yl)methoxy)carbonyl)amino)-3-(naphthalen-2-yl)propanoic acid (Fmoc-3-(2-naphthyl)-L-alanine) (449 mg, 1.02 mmol) as the Fmoc-AA and reacting for 15 hours. The crude was purified by CombiFlash, impurities were washed off the column with 100% EtOAc and desired compound was eluted (DCM:MeOH—100:0 to 90:10). Fractions containing desired product were combined and volatiles removed under reduced pressure to obtain the compound as a white semisolid (352 mg, 0.51 mmol, 50%).

¹H NMR (600 MHz, CDCl₃) δ 7.98 (s, 1H), 7.80-7.73 (m, 3H), 7.67 (s, 1H), 7.52 (t, J=5.9 Hz, 1H), 7.46-7.41 (m, 2H), 7.34 (dd, J=8.4, 1.7 Hz, 1H), 5.95 (s, 1H), 5.88 (d, J=8.1 Hz, 1H), 4.30-4.26 (m, 1H), 4.24-4.16 (m, 1H), 4.14-4.07 (m, 1H), 3.36-3.30 (m, 1H), 3.30-3.23 (m, 1H), 3.13 (dd, J=13.8, 7.8 Hz, 1H), 3.04-2.97 (m, 1H), 2.35-2.25 (m, 2H), 2.08-2.01 (m, 2H), 1.87-1.77 (m, 2H), 1.69-1.61 (m, 1H), 1.57-1.51 (m, 1H), 1.41 (s, 9H), 1.40 (d, J=1.6 Hz, 18H), 1.37-1.31 (m, 1H), 0.92-0.75 (m, 2H). ¹³C NMR (151 MHz, CDCl₃) δ 173.2, 172.9, 172.6, 157.8, 133.5, 133.3, 132.6, 128.6, 128.4, 127.7, 127.7, 127.3, 126.3, 125.9, 82.1, 81.5, 80.7, 53.3, 53.1, 39.4, 38.9, 31.8, 31.7, 31.6, 28.5, 28.3, 28.1, 28.0, 22.1. MS (ESI) m/z: 685.5 [M+H]⁺ (10% MeOH in DCM, with tailing)=0.34

### di-tert-butyl (((S)-6-((S)-2-amino-3-(2-iodophenyl)propanamido)-1-(tert-butoxy)-1-oxohexan-2-yl)carbamoyl)-L-glutamate

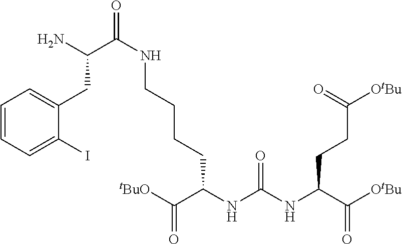 [SMILES: CC(C)(C)OC(=O)CC[C@H](NC(=O)N[C@@H](CCCCNC(=O)[C@@H](N)Cc1ccccc1I)C(=O)OC(C)(C)C)C(=O)OC(C)(C)C]

di-tert-butyl (((S)-6-((S)-2-amino-3-(2-iodophenyl)propanamido)-1-(tert-butoxy)-1-oxohexan-2-yl)carbamoyl)-L-glutamate was prepared from di-tert-butyl (((S)-6-amino-1-(tert-butoxy)-1-oxohexan-2-yl)carbamoyl)-L-glutamate (250 mg, 0.51 mmol) following general procedure I employing (S)-2-((((9H-fluoren-9-yl)methoxy)carbonyl)amino)-3-(2-iodophenyl)propanoic acid (Fmoc-2-iodo-L-phenylalanine) (263 mg, 0.51 mmol) as the Fmoc-AA and reacting for 5 hours. The crude was purified by CombiFlash, impurities were washed off the column with 100% EtOAc and desired compound was eluted (DCM:MeOH—100:0 to 90:10). Fractions containing desired product were combined and volatiles removed under reduced pressure to obtain the compound as a yellowish oil (80 mg, 0.10 mmol, 21%).

¹H NMR (400 MHz, CDCl₃) δ 7.83 (d, J=7.9 Hz, 1H), 7.32-7.15 (m, 2H), 6.92 (td, J=7.4, 2.1 Hz, 1H), 5.46-5.34 (m, 2H), 4.31 (m, 3H), 3.67 (m, 1H), 3.49-3.36 (m, 1H), 3.25 (m, 2H), 2.86 (dd, J=14.0, 7.1 Hz, 1H), 2.40-2.20 (m, 2H), 2.11-2.00 (m, 1H), 1.85-1.70 (m, 3H), 1.74-1.57 (m, 4H), 1.56-1.30 (m, 31H). ¹³C NMR (101 MHz, CDCl₃) δ 174.1, 172.58, 172.56, 172.4, 157.2, 141.2, 139.9, 130.83, 130.79, 128.68, 128.63, 128.60, 101.4, 82.0, 81.7, 80.6, 55.9, 53.5, 53.1, 45.6, 38.7, 32.4, 31.8, 29.1, 28.6, 28.2, 22.4. MS (ESI) m/z: 761.3 [M+H]⁺ R_{f} (10% MeOH in DCM, with tailing)=0.39

### di-tert-butyl (((S)-6-((S)-2-((1r,4S)-4-(aminomethyl)cyclohexane-1-carboxamido)-3-(naphthalen-2-yl)propanamido)-1-(tert-butoxy)-1-oxohexan-2-yl)carbamoyl)-L-glutamate

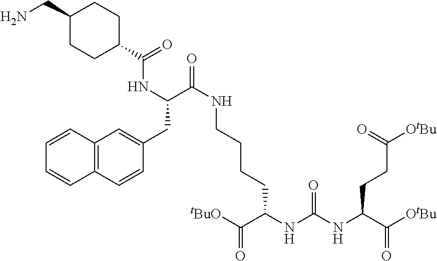 [SMILES: CC(C)(C)OC(=O)CC[C@H](NC(=O)N[C@@H](CCCCNC(=O)[C@H](Cc1ccc2ccccc2c1)NC(=O)[C@H]1CC[C@H](CN)CC1)C(=O)OC(C)(C)C)C(=O)OC(C)(C)C]

di-tert-butyl (((S)-6-((S)-2-((1r,4S)-4-(aminomethyl)cyclohexane-1-carboxamido)-3-(naphthalen-2-yl)propanamido)-1-(tert-butoxy)-1-oxohexan-2-yl)carbamoyl)-L-glutamate was prepared from di-tert-butyl (((S)-6-((S)-2-amino-3-(naphthalen-2-yl)propanamido)-1-(tert-butoxy)-1-oxohexan yl)carbamoyl)-L-glutamate (180 mg, 0.26 mmol) following general procedure I employing (1r,4r)-4-(((((9H-fluoren-9-yl)methoxy)carbonyl)amino)methyl)cyclohexane-1-carboxylic acid (Fmoc-tranexamic acid) (100 mg, 0.26 mmol) as the Fmoc-AA and reacting for 15 hours. The crude was purified by CombiFlash, impurities were washed off the column with 100% EtOAc and desired compound was eluted (DCM:MeOH—100:0 to 90:10). Fractions containing desired product were combined and volatiles removed under reduced pressure to obtain the compound as a yellowish oil (85 mg, 0.10 mmol, 40%).

¹H NMR (400 MHz, CDCl₃) δ 8.45 (s, 1H), 7.83 (d, J=8.3 Hz, 1H), 7.67-7.61 (m, 2H), 7.54 (d, J=8.1 Hz, 1H), 7.42 (t, J=7.5 Hz, 1H), 7.34 (t, J=7.4 Hz, 1H), 7.18-7.07 (m, 2H), 7.01 (d, 1H), 6.17 (d, J=8.7 Hz, 1H), 5.21-5.16 (m, 1H), 4.68-4.57 (m, 1H), 4.32-4.22 (m, 1H), 3.55-3.41 (m, 2H), 3.20 (dd, J=13.8, 4.1 Hz, 1H), 3.13-2.98 (m, 2H), 2.49-2.30 (m, 3H), 2.24-2.10 (m, 1H), 1.97-1.79 (m, 2H), 1.79-1.68 (m, 2H), 1.57 (s, 8H), 1.46-1.35 (m, 27H), 1.20-1.06 (m, 3H), 0.99-0.60 (m, 4H). ¹³C NMR (101 MHz, CDCl₃) δ 177.26, 174.82, 174.20, 172.52, 157.56, 135.06, 133.35, 132.20, 128.05, 127.85, 127.30, 125.91, 125.36, 82.46, 80.93, 80.39, 54.60, 53.26, 52.28, 50.34, 48.12, 44.63, 40.42, 39.67, 39.45, 32.77, 31.65, 29.98, 29.08, 28.90, 28.21, 28.16, 28.07, 26.90, 25.68, 24.71, 23.42. MS (ESI) m/z: 825.7 [M+H]⁺ (10% MeOH in DCM, with tailing)=0.28

### di-tert-butyl (((S)-6-((S)-2-((1r,4S)-4-(aminomethyl)cyclohexane-1-carboxamido)-3-(2-iodophenyl)propanamido)-1-(tert-butoxy)-1-oxohexan-2-yl)carbamoyl)-L-glutamate

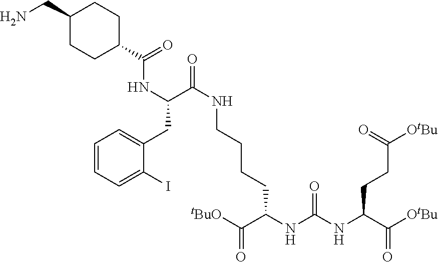 [SMILES: CC(C)(C)OC(=O)CC[C@H](NC(=O)N[C@@H](CCCCNC(=O)[C@H](Cc1ccccc1I)NC(=O)[C@H]1CC[C@H](CN)CC1)C(=O)OC(C)(C)C)C(=O)OC(C)(C)C]

di-tert-butyl (((S)-6-((S)-2-((1r,4S)-4-(aminomethyl)cyclohexane-1-carboxamido)-3-(2-iodophenyl)propanamido)-1-(tert-butoxy)-1-oxohexan-2-yl)carbamoyl)-L-glutamate was prepared from di-tert-butyl (((S)-6-((S)-2-amino-3-(2-iodophenyl)propanamido)-1-(tert-butoxy)-1-oxohexan-2-yl)carbamoyl)-L-glutamate (75 mg, 0.10 mmol) following general procedure I employing (1r,4r)-4-(((((9H-fluoren-9-yl)methoxy)carbonyl)amino)methyl)cyclohexane carboxylic acid (Fmoc-tranexamic acid) (38 mg, 0.10 mmol) as the Fmoc-AA and reacting for 7 hours. The crude was purified by CombiFlash, impurities were washed off the column with 100% EtOAc and desired compound was eluted (DCM:MeOH—100:0 to 90:10). Fractions containing desired product were combined and volatiles removed under reduced pressure to obtain the compound as a white semisolid (70 mg, 0.08 mmol, 76%). R_{f} (10% MeOH in DCM, with tailing)=0.25

¹H NMR (400 MHz, CDCl₃) δ 7.74 (dd, J=7.8, 2.7 Hz, 1H), 7.30 (d, J=7.2 Hz, 1H), 7.19 (t, J=7.6 Hz, 1H), 6.87 (td, J=7.6, 1.7 Hz, 1H), 4.87 (q, J=7.8 Hz, 1H), 4.46-4.31 (m, 1H), 4.24-4.13 (m, 1H), 3.44-3.26 (m, 1H), 3.24-3.10 (m, 2H), 3.05 (q, J=7.3 Hz, 3H), 3.00-2.89 (m, 1H), 2.88-2.74 (m, 2H), 2.43-2.24 (m, 2H), 2.21-2.02 (m, 2H), 1.97-1.81 (m, 3H), 1.81-1.60 (m, 5H), 1.60-1.48 (m, 2H), 1.48-1.39 (m, 27H), 1.28-1.10 (m, 1H), 1.07-0.91 (m, 2H). MS (ESI) m/z: 900.2 [M+H]⁺ (10% MeOH in DCM, with tailing)=0.25

### di-tert-butyl (((S)-6-((S)-2-((S)-2-amino-3-(4-iodophenyl)propanamido)-3-(naphthalen-2-yl)propanamido)-1-(tert-butoxy)-1-oxohexan-2-yl)carbamoyl)-L-glutamate

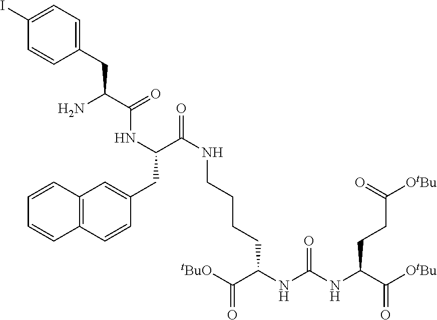 [SMILES: CC(C)(C)OC(=O)CC[C@H](NC(=O)N[C@@H](CCCCNC(=O)[C@H](Cc1ccc2ccccc2c1)NC(=O)[C@@H](N)Cc1ccc(I)cc1)C(=O)OC(C)(C)C)C(=O)OC(C)(C)C]

di-tert-butyl (((S)-6-((S)-2-((S)-2-amino-3-(4-iodophenyl)propanamido)-3-(naphthalen-2-yl)propanamido)-1-(tert-butoxy)-1-oxohexan-2-yl)carbamoyl)-L-glutamate was prepared from di-tert-butyl (((S)-6-((S)-2-amino-3-(naphthalen-2-yl)propanamido)-1-(tert-butoxy)-1-oxohexan-2-yl)carbamoyl)-L-glutamate (40 mg, 0.58 mmol) following general procedure/employing (S)-2-((((9H-fluoren-9-yl)methoxy)carbonyl)amino)-3-(4-iodophenyl)propanoic acid (Fmoc-4-iodo-L-phenylalanine) (30 mg, 0.58 mmol) as the Fmoc-AA and reacting for 17 hours. The crude was purified by CombiFlash, impurities were washed off the column with 100% EtOAc and desired compound was eluted (DCM:MeOH—100:0 to 90:10). Fractions containing desired product were combined and volatiles removed under reduced pressure to obtain the compound as a white semisolid (20 mg, 0.02 mmol, 37%).

¹H NMR (600 MHz, CDCl₃) δ 7.79-7.68 (m, 4H), 7.65 (d, J=6.9 Hz, 1H), 7.43 (dd, J=6.3, 3.4 Hz, 3H), 7.37 (d, J=7.8 Hz, 2H), 7.34 (s, 1H), 6.68 (d, J=8.0, 4.3 Hz, 1H), 6.01 (s, 1H), 4.81 (s, 1H), 4.40-4.29 (m, 1H), 4.27-4.18 (m, 1H), 3.89-3.66 (m, 1H), 3.35 (dd, J=13.7, 6.0 Hz, 2H), 3.27 (q, J=7.3, 5.5 Hz, 1H), 3.23-3.14 (m, 1H), 3.05-2.95 (m, 1H), 2.90 (dd, J=13.9, 5.6 Hz, 1H), 2.70-2.50 (m, 1H), 2.37-2.29 (m, 2H), 2.12-2.01 (m, 1H), 1.90-1.76 (m, 2H), 1.62 (dt, J=33.8, 9.4 Hz, 3H), 1.46-1.34 (m, 27H), 1.31-1.18 (m, 4H). MS (ESI) m/z: 958.4 [M+H]⁺ R_{f} (10% MeOH in DCM, with tailing)=0.27

### di-tert-butyl (((S)-6-((S)-2-((1S,4S)-4-(((S)-2-amino-3-(4-iodophenyl)propanamido)methyl)cyclohexane-1-carboxamido)-3-(naphthalen-2-yl)propanamido)-1-(tert-butoxy)-1-oxohexan-2-yl)carbamoyl)-L-glutamate

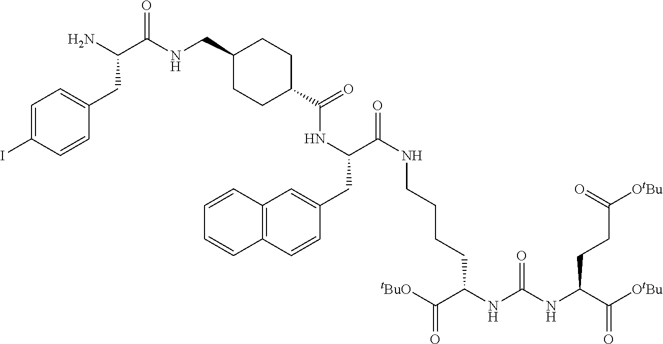 [SMILES: CC(C)(C)OC(=O)CC[C@H](NC(=O)N[C@@H](CCCCNC(=O)[C@H](Cc1ccc2ccccc2c1)NC(=O)[C@H]1CC[C@H](CNC(=O)[C@@H](N)Cc2ccc(I)cc2)CC1)C(=O)OC(C)(C)C)C(=O)OC(C)(C)C]

di-tert-butyl (((S)-6-((S)-2-((1S,4S)-4-(((S)-2-amino-3-(4-iodophenyl)propanamido)methyl)cyclohexane-1-carboxamido)-3-(naphthalen-2-yl)propanamido)-1-(tert-butoxy)-1-oxohexan-2-yl)carbamoyl)-L-glutamate was prepared from di-tert-butyl (((S)-6-((S)-2-((1r,4S)-4-(aminomethyl)cyclohexane-1-carboxamido)-3-(naphthalen-2-yl)propanamido)-1-(tert-butoxy)-1-oxohexan-2-yl)carbamoyl)-L-glutamate (100 mg, 0.12 mmol) following general procedure I employing (S)-2-((((9H-fluoren-9-yl)methoxy)carbonyl)amino)-3-(4-iodophenyl)propanoic acid (Fmoc-4-iodo-L-alanine) (62 mg, 0.12 mmol) as the Fmoc-AA and reacting for 16 hours. Compound was purified by CombiFlash (Heptane:EtOAc:DCM:MeOH—100:0:0:0 to 0:100:0:0 and then 0:0:100:0 to 0:0:90:10). Fractions containing desired product were combined and volatiles removed under reduced pressure to obtain the compound as a white semisolid (75 mg, 0.07 mmol, 56%).

¹H NMR (400 MHz, CDCl₃) δ 7.72-7.53 (m, 5H), 7.44 (t, 1H), 7.39 (t, 1H), 7.21 (t, J=6.1 Hz, 1H), 7.13 (d, J=8.3 Hz, 1H), 6.95 (dd, J=11.7, 8.1 Hz, 2H), 6.12 (d, J=8.5 Hz, 1H), 5.18 (s, 1H), 4.64 (s, 1H), 4.30 (dd, J=9.7, 5.4 Hz, 1H), 3.62-3.38 (m, 4H), 3.22 (dd, J=13.7, 4.3 Hz, 1H), 3.15 (dd, J=13.8, 4.2 Hz, 3H), 3.10-2.99 (m, 4H), 2.98-2.88 (m, 1H), 2.64 (dd, J=13.8, 8.9 Hz, 2H), 2.50-2.32 (m, 4H), 2.27-2.12 (m, 1H), 1.91 (tdd, J=14.8, 8.1, 3.5 Hz, 1H), 1.69 (d, J=40.8 Hz, 2H), 1.59 (s, 8H), 1.46-1.37 (m, 27H), 1.30-1.17 (m, 2H), 1.17-0.95 (m, 1H), 0.93-0.63 (m, 1H). ¹³C NMR (101 MHz, CDCl₃) δ 177.22, 174.96, 173.77, 172.54, 157.58, 137.81, 137.66, 135.08, 133.42, 132.27, 131.50, 131.44, 128.16, 127.96, 127.88, 127.38, 127.07, 126.03, 125.47, 92.19, 82.60, 81.02, 80.48, 56.38, 54.67, 53.34, 52.33, 45.16, 44.52, 40.51, 37.51, 37.07, 31.97, 30.19, 29.65, 29.11, 28.90, 28.29, 28.20, 28.14, 27.96, 26.98, 24.78, 22.78. MS (ESI) m/z: 1098.3 [M+H]⁺ R_{f} (10% MeOH in DCM, with tailing)=0.21

### (S)-2-((1r,4S)-4-(((tert-butoxycarbonyl)amino)methyl)cyclohexane-1-carboxamido)-3-(4-iodophenyl)propanoic acid

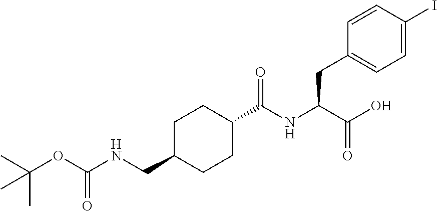 [SMILES: CC(C)(C)OC(=O)NC[C@H]1CC[C@H](C(=O)N[C@@H](Cc2ccc(I)cc2)C(=O)O)CC1]

(S)-2-((1r,4S)-4-(((tert-butoxycarbonyl)amino)methyl)cyclohexane-1-carboxamido)-3-(4-iodophenyl)propanoic acid was prepared following general procedure II, employing (S)-2-amino-3-(4-iodophenyl)propanoic acid (4-iodo-L-phenylalanine) as the alpha-amino acid (390 mg, 0.74 mmol, 86%)

¹H NMR (600 MHz, CD₃OD) δ 7.61 (dt, J=8.3, 1.9 Hz, 2H), 7.01 (dt, J=8.3, 1.8 Hz, 2H), 4.64 (q, J=9.2 Hz, 1H), 3.17 (dd, J=14.0, 5.0 Hz, 1H), 2.93-2.85 (m, 4H), 2.12 (tt, J=12.2, 3.5 Hz, 1H), 1.84-1.74 (m, 5H), 1.67-1.60 (m, 1H), 1.43 (s, 9H), 0.99-0.88 (m, 2H). MS (ESI) m/z: 529.2 [M−H]⁻

### (S)-2-((1r,4S)-4-(((tert-butoxycarbonyl)amino)methyl)cyclohexane-1-carboxamido)-3-(3-iodophenyl)propanoic acid

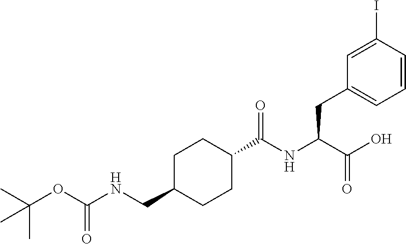 [SMILES: CC(C)(C)OC(=O)NC[C@H]1CC[C@H](C(=O)N[C@@H](Cc2cccc(I)c2)C(=O)O)CC1]

(S)-2-((1r,4S)-4-(((tert-butoxycarbonyl)amino)methyl)cyclohexane-1-carboxamido)-3-(3-iodophenyl)propanoic acid was prepared following general procedure II, employing (S)-2-amino-3-(3-iodophenyl)propanoic acid (3-iodo-L-phenylalanine) as the alpha-amino acid (390 mg, 0.74 mmol, 86%)

¹H NMR (400 MHz, CD₃OD) 7.61 (dt, J=8.3, 1.9 Hz, 2H), 7.01 (dt, J=8.3, 1.8 Hz, 2H), 4.64 (q, J=9.2 Hz, 1H), 3.17 (dd, J=14.0, 5.0 Hz, 1H), 2.93-2.85 (m, 4H), 2.12 (tt, J=12.2, 3.5 Hz, 1H), 1.84-1.74 (m, 5H), 1.67-1.60 (m, 1H), 1.43 (s, 9H), 0.99-0.88 (m, 2H). ¹³C NMR (101 MHz, MeOD) δ 178.7, 174.4, 158.6, 141.2, 139.5, 136.9, 131.2, 129.7, 94.8, 79.8, 54.3, 46.0, 39.0, 37.8, 30.8, 29.8, 28.8, 26.3. MS (ESI) m/z: 529.3 [M−H]⁻

### (R)-2-((1r,4R)-4-(((tert-butoxycarbonyl)amino)methyl)cyclohexane-1-carboxamido)-3-(5-iodo-1H-indol-3-yl)propanoic acid

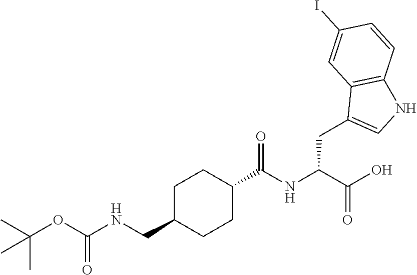 [SMILES: CC(C)(C)OC(=O)NC[C@H]1CC[C@H](C(=O)N[C@H](Cc2c[nH]c3ccc(I)cc23)C(=O)O)CC1]

(R)-2-((1r,4R)-4-(((tert-butoxycarbonyl)amino)methyl)cyclohexane-1-carboxamido)-3-(5-iodo-1H-indol-3-yl)propanoic acid was prepared following general procedure II, employing 2-amino-3-(5-iodo-1H-indol-3-yl)propanoic acid as the alpha-amino acid, the compound was purified by CombiFlash (Heptane:EtOAc:AcOH—100:0:0.5 to 15:85:0.5) (100 mg, 0.17 mmol, 46%) R_{f} (40 EtOAc in heptane, with tailing)=0.21

¹H NMR (400 MHz, CD₃OD) δ 10.53 (s, 1H), 7.90 (d, J=1.6 Hz, 1H), 7.34 (dd, J=8.5, 1.6 Hz, 1H), 7.16 (d, J=8.5 Hz, 1H), 7.08 (d, J=2.0 Hz, 1H), 4.70 (dd, J=7.8, 4.8 Hz, 1H), 4.11 (q, J=7.1 Hz, 1H), 3.35-3.26 (m, 2H), 3.14 (dd, J=14.7, 7.9 Hz, 1H), 2.87 (d, J=6.7 Hz, 2H), 2.13 (tt, J=12.2, 3.5 Hz, 1H), 1.86-1.73 (m, 3H), 1.74-1.65 (m, 1H), 1.44 (s, 9H), 1.02-0.86 (m, 2H). ¹³C NMR (101 MHz, CD₃OD) δ 178.7, 175.0, 158.7, 137.1, 131.8, 130.7, 128.4, 125.7, 114.5, 110.9, 82.8, 79.8, 54.6, 46.0, 39.1, 30.9, 30.0, 28.8, 28.2. MS (ESI) m/z: 568.2 [M−H]⁻

### di-tert-butyl (((S)-1-(tert-butoxy)-6-((S)-2-((1r,4S)-4-(((tert-butoxycarbonyl)amino)methyl)cyclohexane-1-carboxamido)-3-(4-iodophenyl)propanamido)-1-oxohexan-2-yl)carbamoyl)-L-glutamate

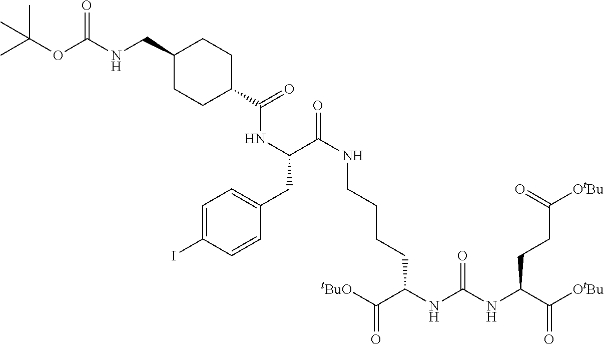 [SMILES: CC(C)(C)OC(=O)CC[C@H](NC(=O)N[C@@H](CCCCNC(=O)[C@H](Cc1ccc(I)cc1)NC(=O)[C@H]1CC[C@H](CNC(=O)OC(C)(C)C)CC1)C(=O)OC(C)(C)C)C(=O)OC(C)(C)C]

di-tert-butyl (((S)-1-(tert-butoxy)-6-((S)-2-((1r,4S)-4-(((tert-butoxycarbonyl)amino)methyl)cyclohexane-1-carboxamido)-3-(4-iodophenyl)propanamido)-1-oxohexan-2-yl)carbamoyl)-L-glutamate was prepared following general procedure III, employing (S)-2-((1r,4S)-4-(((tert-butoxycarbonyl)amino)methyl)cyclohexane-1-carboxamido) (4-iodophenyl)propanoic acid as the carboxylic acid and reacting for 5 hours. Compound was obtained as a white/yellow solid (296 mg, 0.29 mmol, 63%). ¹H NMR (400 MHz, CDCl₃, mixture of un-assigned rotamers) δ 7.56 (d, J=8.0 Hz, 2H), 7.48 (d, J=7.9 Hz, 2H), 6.96-6.86 (m, 2H), 6.67-6.60 (m, 1H), 6.01-5.97 (m, 1H), 5.63-5.57 (m, 1H), 5.48-5.40 (m, 1H), 4.97 (s, 1H), 4.70-4.50 (m, 1H), 4.39-4.17 (m, 2H), 3.28-3.14 (m, 1H), 3.09-2.95 (m, 1H), 2.98-2.86 (m, 4H), 2.89-2.77 (m, 1H), 2.43-2.21 (m, 2H), 2.16-1.98 (m, 1H), 2.05-2.01 (m, 1H), 1.98-1.94 (m, 2H), 1.92-1.68 (m, 1H), 1.81-1.77 (m, 7H), 1.47-1.40 (m, 36H), 1.38-1.29 (m, 1H), 1.28-1.20 (m, 1H), 0.98-0.75 (m, 2H). ¹³C NMR (101 MHz, CDCl₃, mixture of un-assigned rotamers) δ 176.23, 172.92, 172.70, 172.66, 172.56, 172.50, 171.54, 162.70, 157.66, 157.54, 157.32, 157.28, 156.23, 137.61, 137.44, 136.69, 131.55, 131.44, 92.27, 92.04, 82.57, 82.29, 82.21, 81.82, 81.69, 81.61, 81.09, 80.75, 80.55, 79.18, 77.36, 60.51, 54.35, 54.29, 53.48, 53.26, 53.17, 53.04, 52.43, 46.71, 45.48, 45.14, 44.65, 38.63, 38.18, 36.61, 32.58, 32.00, 31.78, 31.75, 29.98, 29.85, 29.77, 29.44, 29.20, 29.12, 28.86, 28.74, 28.56, 28.45, 28.24, 28.21, 28.15, 22.55, 22.14 MS (ESI) m/z: 1001.5 [M+H]⁺ R_{f} (60% EtOAc in heptane)=0.32

### di-tert-butyl (((S)-1-(tert-butoxy)-6-((S)-2-((1r,4S)-4-(((tert-butoxycarbonyl)amino)methyl)cyclohexane-1-carboxamido)-3-(3-iodophenyl)propanamido)-1-oxohexan-2-yl)carbamoyl)-L-glutamate

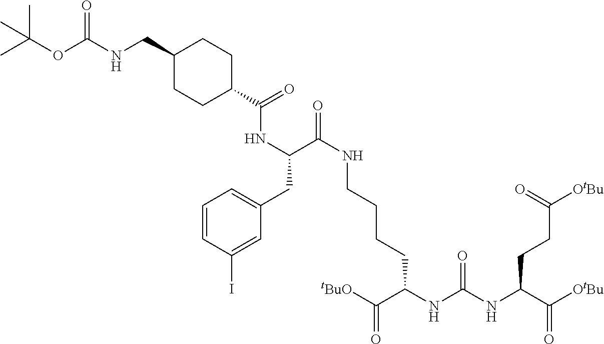 [SMILES: CC(C)(C)OC(=O)CC[C@H](NC(=O)N[C@@H](CCCCNC(=O)[C@H](Cc1cccc(I)c1)NC(=O)[C@H]1CC[C@H](CNC(=O)OC(C)(C)C)CC1)C(=O)OC(C)(C)C)C(=O)OC(C)(C)C]

di-tert-butyl (((S)-1-(tert-butoxy)-6-((S)-2-((1r,4S)-4-(((tert-butoxycarbonyl)amino)methyl)cyclohexane-1-carboxamido)-3-(3-iodophenyl)propanamido)-1-oxohexan-2-yl)carbamoyl)-L-glutamate was prepared following general procedure III, employing (S)-2-((1r,4S)-4-(((tert-butoxycarbonyl)amino)methyl)cyclohexane-1-carboxamido)-3-(3-iodophenyl)propanoic acid as the carboxylic acid and reacting for 24 hours. Compound was obtained as an off-white solid (210 mg, 0.21 mmol, 56%). ¹H NMR (400 MHz, CDCl₃, mixture of un-assigned rotamers) δ 7.59-7.47 (m, 2H), 7.05-6.94 (m, 1H), 6.70-6.62 (m, 1H), 6.03-5.97 (m, 1H), 4.94-4.90 (m, 1H), 4.67-4.55 (m, 2H), 4.35-4.23 (m, 1H), 3.51-3.43 (m, 1H), 3.03-2.85 (m, 4H), 2.81-2.77 (m, 7H), 2.45-2.26 (m, 2H), 2.22-1.98 (m, 1H), 1.92-1.70 (m, 2H), 1.69-1.61 (m, 1H), 1.58-1.54 (m, 6H), 1.50-1.39 (m, 38H), 1.36-1.20 (m, 1H), 0.98-0.84 (m, 1H), 0.84-0.77 (m, 1H). ¹³C NMR (101 MHz, CDCl₃, mixture of un-assigned rotamers) δ 177.36, 176.09, 175.08, 172.63, 172.58, 172.54, 172.49, 157.56, 157.26, 157.24, 156.15, 140.08, 139.54, 138.47, 138.28, 135.86, 130.32, 130.18, 128.73, 128.41, 94.17, 82.80, 82.22, 82.12, 81.78, 81.63, 81.04, 80.67, 80.48, 79.16, 77.36, 54.73, 53.42, 53.26, 53.16, 52.35, 46.74, 45.51, 44.66, 38.88, 38.73, 37.57, 31.75, 31.73, 30.07, 29.99, 29.88, 29.69, 29.59, 29.21, 29.07, 28.86, 28.55, 28.47, 28.41, 28.24, 28.21, 28.17, 28.15. MS (ESI) m/z: 1001.5 [M+H]⁺ R_{f} (60% EtOAc in heptane)=0.38

### di-tert-butyl (((S)-1-(tert-butoxy)-6-((R)-2-((1r,4R)-4-(((tert-butoxycarbonyl)amino)methyl)cyclohexane-1-carboxamido)-3-(5-iodo-1H-indol-3-yl)propanamido)-1-oxohexan-2-yl)carbamoyl)-L-glutamate

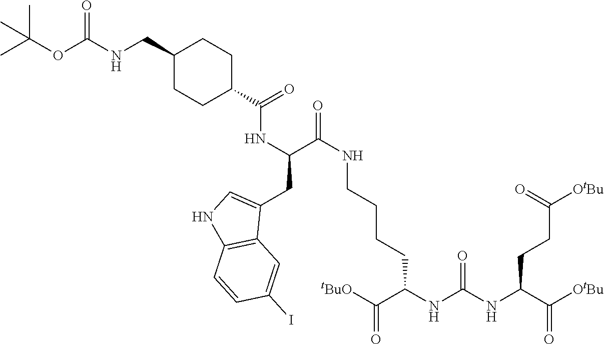 [SMILES: CC(C)(C)OC(=O)CC[C@H](NC(=O)N[C@@H](CCCCNC(=O)[C@@H](Cc1c[nH]c2ccc(I)cc12)NC(=O)[C@H]1CC[C@H](CNC(=O)OC(C)(C)C)CC1)C(=O)OC(C)(C)C)C(=O)OC(C)(C)C]

di-tert-butyl (((S)-1-(tert-butoxy)-6-((R)-2-((1r,4R)-4-(((tert-butoxycarbonyl)amino)methyl)cyclohexane-1-carboxamido)-3-(5-iodo-1H-indol-3-yl)propanamido)-1-oxohexan-2-yl)carbamoyl)-L-glutamate was prepared following general procedure III, employing (R)-2-((1r,4R)-4-(((tert-butoxycarbonyl)amino)methyl)cyclohexane-1-carboxamido)-3-(5-iodo-1H-indol-3-yl)propanoic acid as the carboxylic acid and reacting for 10 hours. Compound was obtained as a white semisolid (80 mg, 0.08 mmol, 45%). ¹H NMR (400 MHz, CDCl₃, mixture of un-assigned rotamers) δ 9.70 (s, 1H), 8.99 (s, 1H), 8.01 (d, J=3.7 Hz, 1H), 7.91 (d, J=1.5 Hz, 1H), 7.44-7.34 (m, 1H), 7.22 (d, J=8.5 Hz, 1H), 7.13 (d, J=8.5 Hz, 1H), 7.02-6.95 (m, 1H), 6.60 (d, J=7.6 Hz, 1H), 6.20 (s, 1H), 5.80 (s, 1H), 5.70 (d, J=8.3 Hz, 1H), 5.42-5.31 (m, 1H), 4.34-4.29 (m, 1H), 4.20-4.12 (m, 1H), 3.16-3.08 (m, 1H), 3.03-2.91 (m, 3H), 2.40-2.27 (m, 2H), 2.10-2.01 (m, 1H), 1.92-1.83 (m, 1H), 1.83-1.80 (m, 2H), 1.79-1.71 (m, 2H), 1.49-1.40 (m, 45H), 1.30-1.21 (m, 3H), 1.21-1.11 (m, 1H), 0.97-0.83 (m, 4H). ¹³C NMR (101 MHz, CDCl₃, mixture of un-assigned rotamers) δ 176.58, 175.89, 173.80, 172.68, 172.65, 172.59, 171.62, 157.76, 157.63, 156.26, 135.60, 135.41, 130.34, 130.16, 130.06, 127.67, 127.56, 124.80, 124.36, 113.82, 113.56, 110.12, 109.74, 82.97, 82.85, 82.36, 82.33, 81.95, 81.44, 80.90, 80.71, 79.23, 77.36, 60.52, 54.12, 53.90, 53.58, 53.45, 53.31, 52.86, 46.74, 45.22, 44.75, 39.05, 38.76, 37.79, 32.32, 31.87, 31.84, 29.93, 29.75, 29.21, 28.69, 28.57, 28.45, 28.22, 28.16, 28.14, 22.55, 21.16. MS (ESI) m/z: 1040.6 [M+H]⁺ R_{f} (60% EtOAc in heptane)=0.33

### ((1S,4r)-4-(((S)-1-(((S)-5-carboxy-5-(3-((S)-1,3-dicarboxypropyl)ureido)pentyl)amino)-3-(4-iodophenyl)-1-oxopropan-2-yl)carbamoyl)cyclohexyl)methanaminium trifluoroacetate

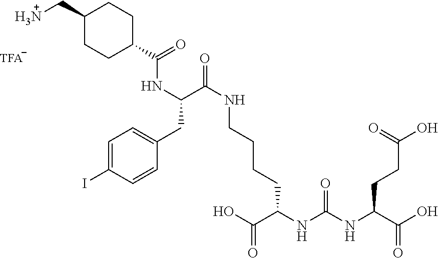 [SMILES: [NH3+]C[C@H]1CC[C@H](C(=O)N[C@@H](Cc2ccc(I)cc2)C(=O)NCCCC[C@H](NC(=O)N[C@@H](CCC(=O)O)C(=O)O)C(=O)O)CC1]

((1S,4r))-4-(((S)-1-(((S)-5-carboxy-5-(3-((S)-1,3-dicarboxypropyl)ureido)pentyl)amino)-3-(4-iodophenyl)-1-oxopropan-2-yl)carbamoyl)cyclohexyl)methanaminium trifluoroacetate was obtained from di-tert-butyl (((S)-1-(tert-butoxy)-6-((S)-2-((1r,4S)-4-(((tert-butoxycarbonyl)amino)methyl)cyclohexane-1-carboxamido)-3-(4-iodophenyl)propanamido)-1-oxohexan-2-yl)carbamoyl)-L-glutamate, following general procedure IV, as a viscous oil (TFA salt—248 mg, 0.29 mmol, 99%)

¹H NMR (400 MHz, (CD₃)₂SO) δ 7.96-7.85 (m, 2H), 7.74-7.65 (m, 4H), 7.60 (d, J=7.9 Hz, 2H), 7.02 (d, J=7.9 Hz, 2H), 6.31 (t, J=9.6 Hz, 2H), 4.47-4.36 (m, 1H), 4.14-3.98 (m, 3H), 3.07-2.93 (m, 3H), 2.92-2.84 (m, 1H), 2.76-2.59 (m, 3H), 2.34-2.16 (m, 2H), 2.12-2.00 (m, 1H), 1.98-1.85 (m, 1H), 1.81-1.61 (m, 5H), 1.57-1.42 (m, 1H), 1.41-1.20 (m, 6H), 1.17-1.00 (m, 1H), 0.98-0.81 (m, 2H). ¹³C NMR (101 MHz, (CD₃)₂SO) δ 174.6, 174.5, 174.1, 173.7, 170.8, 157.3, 137.9, 136.6, 131.7, 91.9, 53.3, 52.3, 51.7, 44.3, 43.2, 35.0, 31.7, 29.9, 28.9, 28.8, 28.7, 28.3, 28.0, 27.5, 22.6. MS (ESI) m/z: 732.4 [M−H]⁺

### ((1S,4r)-4-(((S)-1-(((S)-5-carboxy-5-(3-((S)-1,3-dicarboxypropyl)ureido)pentyl)amino)-3-(3-iodophenyl)-1-oxopropan-2-yl)carbamoyl)cyclohexyl)methanaminium trifluoroacetate

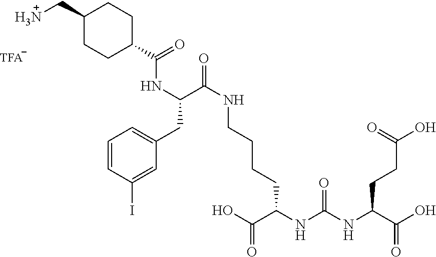 [SMILES: [NH3+]C[C@H]1CC[C@H](C(=O)N[C@@H](Cc2cccc(I)c2)C(=O)NCCCC[C@H](NC(=O)N[C@@H](CCC(=O)O)C(=O)O)C(=O)O)CC1]

((1S,4r))-4-MS)-1-MS)-5-carboxy-5-(3-((S)-1,3-dicarboxypropyl)ureido)pentyl)amino) iodophenyl)-1-oxopropan-2-yl)carbamoyl)cyclohexyl)methanaminium trifluoroacetate was obtained from di-tert-butyl (((S)-1-(tert-butoxy)-6-((S)-2-((1r,4S)-4-(((tert-butoxycarbonyl)amino)methyl)cyclohexane-1-carboxamido)-3-(3-iodophenyl)propanamido) oxohexan-2-yl)carbamoyl)-L-glutamate, following general procedure IV, as a transparent oil (TFA salt—116 mg, 0.14 mmol, 94%)¹H NMR (400 MHz, D₂O) δ 7.63-7.53 (m, 2H), 7.22 (d, J=7.7 Hz, 1H), 7.08 (t, J=7.8 Hz, 1H), 4.46 (q, J=7.8 Hz, 1H), 4.26 (dd, J=8.9, 5.2 Hz, 1H), 4.22-4.08 (m, 1H), 3.17 (q, J=6.3 Hz, 2H), 3.05-2.89 (m, 3H), 2.89-2.78 (m, 3H), 2.49 (q, J=7.5 Hz, 3H), 2.27-2.08 (m, 2H), 2.02-1.90 (m, 1H), 1.91-1.77 (m, 5H), 1.77-1.70 (m, 2H), 1.66-1.58 (m, 2H), 1.45-1.27 (m, 5H), 1.27-1.13 (m, 2H), 1.12-0.94 (m, 3H). MS (ESI) m/z: 732.4 [M−H]⁺

### ((1R,4r)-4-(((R)-1-(((S)-5-carboxy-5-(3-((S)-1,3-dicarboxypropyl)ureido)pentyl)amino)-3-(5-iodo-1H-indol-3-yl)-1-oxopropan-2-yl)carbamoyl)cyclohexyl)methanaminium trifluoroacetate

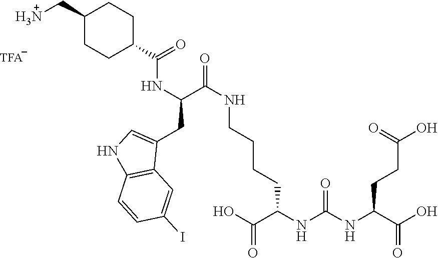 [SMILES: [NH3+]C[C@H]1CC[C@H](C(=O)N[C@H](Cc2c[nH]c3ccc(I)cc23)C(=O)NCCCC[C@H](NC(=O)N[C@@H](CCC(=O)O)C(=O)O)C(=O)O)CC1]

((1R,4r)-4-(((R)-1-(((S)-5-carboxy-5-(3-((S)-1,3-dicarboxypropyl)ureido)pentyl)amino)-3-(5-iodo-1H-indol-3-yl)-1-oxopropan-2-yl)carbamoyl)cyclohexyl)methanaminium trifluoroacetate was obtained from di-tert-butyl (((S)-1-(tert-butoxy)-6-((R)-2-((1 r,4R)-4-(((tert-butoxycarbonyl)amino)methyl)cyclohexane-1-carboxamido)-3-(5-iodo-1H-indol-3-yl)propanamido)-1-oxohexan-2-yl)carbamoyl)-L-glutamate, following general procedure IV. In this case the deprotection cocktail was 1/1 TFA:TIPS:phenol (95:5:5)/DCM. The deprotected compound was purified by preparatory HPLC to obtain the title compound as a transparent oil (TFA salt—23 mg, 0.03 mmol, 34%)

¹H NMR (600 MHz, CD₃OD-d₄) δ 7.93 (s, 1H), 7.33 (d, J=8.2 Hz, 1H), 7.16 (d, J=8.3 Hz, 1H), 7.09 (d, J=5.8 Hz, 1H), 4.58-4.52 (m, 1H), 4.37-4.28 (m, 1H), 4.23-4.15 (m, 1H), 3.21-3.13 (m, 2H), 3.12-2.99 (m, 2H), 2.83-2.73 (m, 2H), 2.48-2.34 (m, 2H), 2.28-2.18 (m, 1H), 2.16-2.07 (m, 1H), 1.87 (q, J=11.6, 9.3 Hz, 5H), 1.78-1.66 (m, 2H), 1.65-1.51 (m, 2H), 1.48-1.30 (m, 3H), 1.30-1.15 (m, 2H), 1.13-0.99 (m, 2H). ¹³C NMR (151 MHz, CD₃OD) δ 178.3, 175.1, 174.8, 174.6, 174.6, 160.0, 137.0, 131.6, 130.7, 128.5, 125.9, 114.5, 110.5, 82.8, 55.8, 54.2, 53.5, 46.3, 45.4, 40.0, 36.7, 32.7, 30.9, 30.3, 29.6, 29.4, 29.1, 28.5, 23.9.

### (((S)-1-carboxy-5-((S)-2-((1S,4S)-4-(((S)-3-(4-iodophenyl)-2-(2-(4,7,10-tris(carboxymethyl)-1,4,7,10-tetraazacyclododecan-1-yl)acetamido)propanamido)methyl)cyclohexane carboxamido)-3-(naphthalen-2-yl)propanamido)pentyl)carbamoyl)-L-glutamic acid trifluoroacetic acid salt (Im)

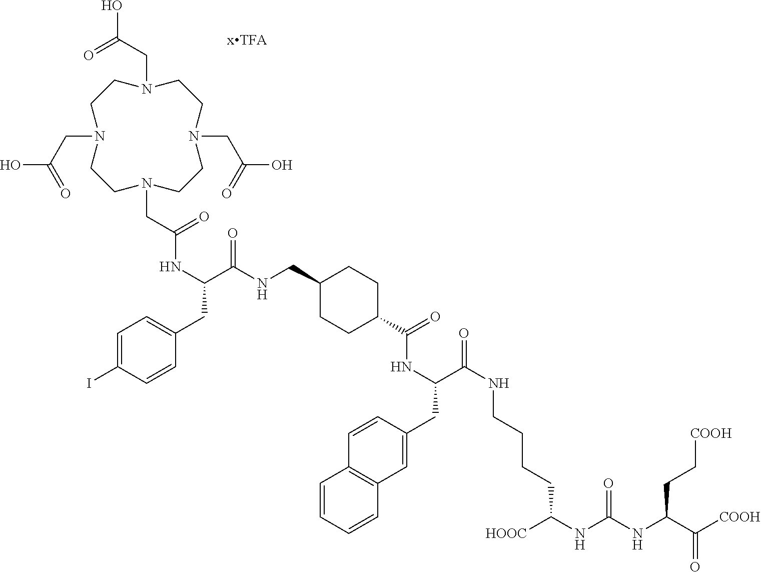 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)[C@H](Cc2ccc(I)cc2)NC(=O)CN2CCN(CC(=O)O)CCN(CC(=O)O)CCN(CC(=O)O)CC2)CC1)C(=O)O)C(=O)C(=O)O]

The title compound was obtained from di-tert-butyl (((S)-6-((S)-2-((1S,4S)-4-(((S)-2-amino-3-(4-iodophenyl)propanamido)methyl)cyclohexane-1-carboxamido)-3-(naphthalen-2-yl)propanamido)-1-(tert-butoxy)-1-oxohexan-2-yl)carbamoyl)-L-glutamate following general procedure V-A. Preparatory HPLC purification was carried out using a gradient of 0% to 100% of B over 15 minutes. Fractions containing the desired compound were lyophilised to obtain Im as white powder. (11 mg, 0.01 mmol, 13%).

¹H NMR (600 MHz, CD₃CN+10% D₂O) δ 7.88-7.76 (m, 3H), 7.72-7.55 (m, 3H), 7.52-7.41 (m, 1H), 7.41-7.34 (m, 1H), 7.07-6.96 (m, 3H), 4.60-4.52 (m, 1H), 4.52-4.42 (m, 1H), 4.25-4.16 (m, 1H), 4.12-4.01 (m, 1H), 3.57 (s, 8H), 3.29-2.70 (m, 18H), 2.42-2.31 (m, 2H), 2.11-1.99 (m, 1H), 1.87-1.73 (m, 1H), 1.72-1.57 (m, 2H), 1.56-1.41 (m, 2H), 1.40-1.27 (m, 2H), 1.27-1.15 (m, 3H), 1.15-1.03 (m, 1H), 0.78-0.61 (m, 4H).

¹³C NMR (151 MHz, CD₃CN+10% D₂O) δ 178.6, 178.51, 176.52, 176.4, 176.2, 175.9, 173.10, 173.07, 172.2, 161.6 (q, J=34.6 Hz), 159.4, 138.5, 138.5, 137.9, 135.9, 134.4, 133.3, 132.80, 132.76, 128.9, 128.8, 128.57, 128.53, 127.2, 126.7, 117.7 (overlapped with CD₃CN signal, q, J=292.9 Hz) 92.6, 55.8, 55.5, 53.8, 53.3, 46.3, 46.1, 45.4, 43.7, 40.8, 39.5, 39.4, 38.6, 38.3, 37.7, 37.5, 31.9, 30.9, 30.3, 30.2, 30.1, 29.71, 29.66, 29.60, 29.5, 29.1, 27.9, 27.6, 25.2, 23.3, 23.1.

### (((S)-1-carboxy-5-((S)-2-((S)-3-(4-iodophenyl)-2-(2-(4,7,10-tris(carboxymethyl)-1,4,7,10-tetraazacyclododecan-1-yl)acetamido)propanamido)-3-(naphthalen-2-yl)propanamido)pentyl)carbamoyl)-L-glutamic acid trifluoroacetic acid salt (In)

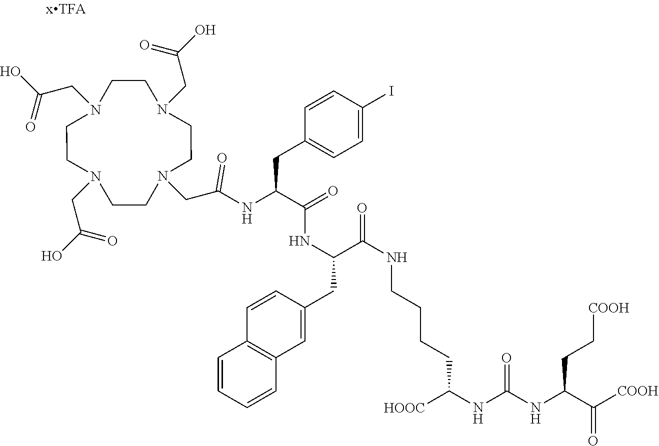 [SMILES: O=C(O)CC[C@H](NC(=O)N[C@@H](CCCCNC(=O)[C@H](Cc1ccc2ccccc2c1)NC(=O)[C@H](Cc1ccc(I)cc1)NC(=O)CN1CCN(CC(=O)O)CCN(CC(=O)O)CCN(CC(=O)O)CC1)C(=O)O)C(=O)C(=O)O]

The title compound was obtained from di-tert-butyl (((S)-6-((S)-2-((S)-2-amino-3-(4-iodophenyl)propanamido)-3-(naphthalen-2-yl)propanamido)-1-(tert-butoxy)-1-oxohexan-2-yl)carbamoyl)-L-glutamate following general procedure V-A. Preparatory HPLC purification was carried out using a gradient of 0% to 100% of B over 15 minutes. Fractions containing the desired compound were lyophilised to obtain In as white powder. (10 mg, 0.01 mmol, 10%).

¹H NMR (600 MHz, CD₃CN+10% D₂O) δ 7.83 (q, J=8.3 Hz, 3H), 7.68 (s, 1H), 7.52 (d, J=7.8 Hz, 2H), 7.46 (q, J=7.5 Hz, 2H), 7.36 (dd, J=8.6, 1.8 Hz, 1H), 6.85 (d, J=7.9 Hz, 2H), 4.61 (t, J=7.5 Hz, 1H), 4.58-4.49 (m, 1H), 4.20 (dd, 1H), 4.06 (dd, J=8.7, 4.9 Hz, 1H), 3.85-3.42 (m, 6H), 3.31 (s, 23H), 3.24-3.08 (m, 1H), 3.08-2.83 (m, 2H), 2.67 (dd, J=13.8, 9.4 Hz, 1H), 2.36 (td, J=7.5, 2.3 Hz, 2H), 2.09-2.01 (m, 1H), 1.88-1.77 (m, 1H), 1.64-1.53 (m, 1H), 1.53-1.42 (m, 1H), 1.33-1.18 (m, 2H), 1.18-1.04 (m, 2H).

¹³C NMR (151 MHz, CD₃CN+10% D₂O) δ 176.34, 176.18, 175.85, 172.69, 172.06, 161.74, 161.51, 161.28, 161.05, 159.37, 138.51, 138.33, 138.17, 135.55, 134.41, 133.39, 132.76, 129.03, 128.97, 128.79, 128.64, 128.60, 127.27, 126.83, 92.43, 55.64, 55.36, 53.74, 53.33, 39.70, 38.86, 37.62, 32.02, 30.92, 28.94, 27.98, 26.69, 26.16, 24.81, 23.20.

### (((S)-1-carboxy-5-((S)-3-(2-iodophenyl)-2-((1r,4S)-4-((2-(4,7,10-tris(carboxymethyl)-1,4,7,10-tetraazacyclododecan-1-yl)acetamido)methyl)cyclohexane-1-carboxamido)propanamido)pentyl)carbamoyl)-L-glutamic acid trifluoroacetic acid salt (Ii)

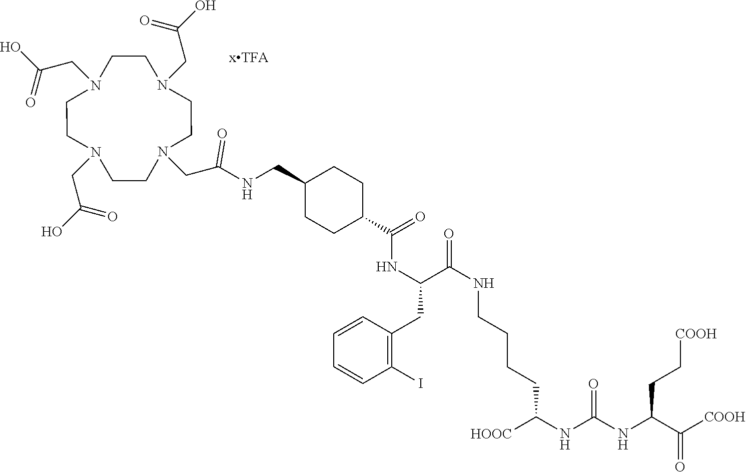 [SMILES: O=C(O)CC[C@H](NC(=O)N[C@@H](CCCCNC(=O)[C@H](Cc1ccccc1I)NC(=O)[C@H]1CC[C@H](CNC(=O)CN2CCN(CC(=O)O)CCN(CC(=O)O)CCN(CC(=O)O)CC2)CC1)C(=O)O)C(=O)C(=O)O]

The title compound was obtained from di-tert-butyl (((S)-6-((S)-2-((1r,4S)-4-(aminomethyl)cyclohexane-1-carboxamido)-3-(2-iodophenyl)propanamido)-1-(tert-butoxy)-1-oxohexan-2-yl)carbamoyl)-L-glutamate following general procedure V-A. Preparatory HPLC purification was carried out using a gradient of 0% to 100% of B over 15 minutes. Fractions containing the desired compound were lyophilised to obtain Ii as white powder. (22 mg, 0.02 mmol, 27%)

¹H NMR (600 MHz, CD₃CN+10% D₂O) δ 7.83 (dt, J=7.7, 1.5 Hz, 1H), 7.32-7.26 (m, 1H), 7.26-7.20 (m, 1H), 6.95 (tt, J=7.4, 1.9 Hz, 1H), 4.55-4.49 (m, 1H), 4.23-4.17 (m, 1H), 4.11-4.06 (m, 1H), 3.72 (d, J=90.6 Hz, 5H), 3.29-2.79 (m, 13H), 2.39-2.29 (m, 3H), 2.13-2.01 (m, 1H), 1.88-1.76 (m, 1H), 1.76-1.66 (m, 3H), 1.66-1.59 (m, 1H), 1.59-1.47 (m, 1H), 1.43-1.29 (m, 2H), 1.29-1.12 (m, 4H), 0.93-0.78 (m, 3H).

¹³C NMR (151 MHz, CD₃CN+10% D₂O) δ 178.5, 178.4, 176.46, 176.43, 176.3, 175.82, 175.79, 172.54, 172.51, 161.46 (q, J=34.7 Hz), 159.4, 140.8, 140.6, 131.9, 129.8, 129.5, 117.7 (overlapped with CD₃CN signal, q, J=292.9 Hz), 101.4, 62.8, 55.9, 54.2, 54.1, 53.96, 53.93, 53.32, 53.28, 46.4, 45.4, 42.9, 39.6, 39.4, 37.7, 32.1, 32.0, 30.9, 30.34, 30.29, 29.61, 29.58, 29.5, 29.2, 29.1, 28.1, 27.9, 23.4, 23.2.

### (((S)-1-carboxy-5-((S)-3-(3-iodophenyl)-2-((1r,4S)-4-((2-(4,7,10-tris(carboxymethyl)-1,4,7,10-tetraazacyclododecan-1-yl)acetamido)methyl)cyclohexane-1-carboxamido)propanamido)pentyl)carbamoyl)-L-glutamic acid trifluoroacetic acid salt (Ij)

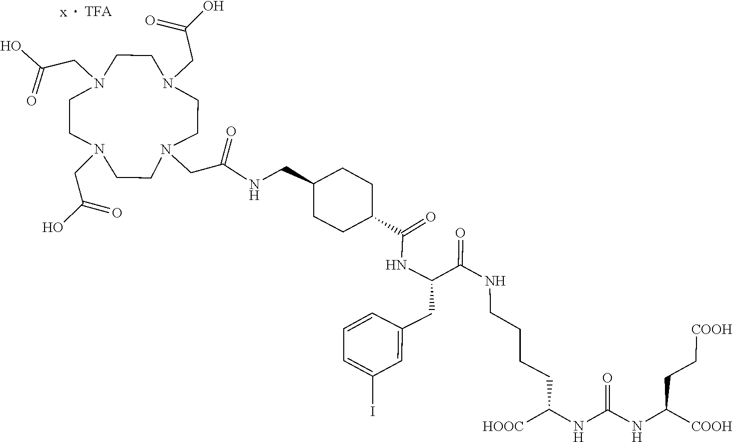

The title compound was obtained from ((1S,4r)-4-(((S)-1-(((S)-5-carboxy-5-(3-((S)-1,3-dicarboxypropyl)ureido)pentyl)amino)-3-(3-iodophenyl)-1-oxopropan-2-yl)carbamoyl)cyclohexyl)methanaminium trifluoroacetate following general procedure V-B. Preparatory HPLC purification was carried out using a method consisting of 8 min of 100% A after injection followed by a gradient from 0 to 100% B over 20 min. Fractions containing the desired compound were lyophilised to obtain Ij as white powder. (24 mg, 0.02 mmol, 30%)

¹H NMR (600 MHz, CD₃CN+10% D₂O) δ 7.59-7.54 (m, 2H), 7.22 (ddt, J=7.8, 2.9, 1.3 Hz, 1H), 7.08-7.02 (m, 1H), 4.45-4.38 (m, 1H), 4.22-4.17 (m, 1H), 4.14-4.07 (m, 1H), 3.89-3.54 (m, 5H), 3.37-2.89 (m, 20H), 2.79 (dd, J=13.8, 9.0 Hz, 1H), 2.37 (ddt, J=8.2, 6.5, 1.6 Hz, 2H), 2.12-2.01 (m, 2H), 1.89-1.77 (m, 1H), 1.76-1.66 (m, 4H), 1.64-1.53 (m, 2H), 1.44-1.32 (m, 3H), 1.32-1.17 (m, 4H), 0.96-0.84 (m, 2H).

¹³C NMR (151 MHz, CD₃CN+10% D₂O) δ 178.6, 178.5, 176.50, 176.48, 176.3, 175.88, 175.86, 172.73, 172.70, 161.6 (q, J=34.5 Hz), 159.4, 141.0, 139.2, 136.7, 131.4, 129.8, 117.7 (overlapped with CD₃CN signal, q, J=293.2 Hz), 94.7, 55.9, 55.3, 55.2, 53.93, 53.88, 53.31, 53.28, 46.4, 45.39, 45.37, 39.6, 39.4, 37.9, 37.7, 32.1, 32.0, 30.4, 30.3, 29.79, 29.77, 29.4, 29.2, 29.1, 27.98, 27.94, 23.4, 23.2.

### (((S)-1-carboxy-5-((S)-3-(4-iodophenyl)-2-((1r,4S)-4-((2-(4,7,10-tris(carboxymethyl)-1,4,7,10-tetraazacyclododecan-1-yl)acetamido)methyl)cyclohexane-1-carboxamido)propanamido)pentyl)carbamoyl)-L-glutamic acid trifluoroacetic acid salt (Ik)

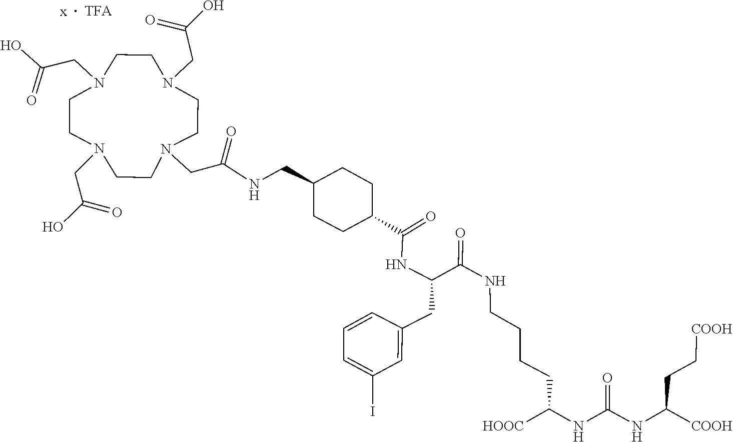

The title compound was obtained from ((1S,4r)-4-(((S)-1-(((S)-5-carboxy-5-(3-((S)-1,3-dicarboxypropyl)ureido)pentyl)amino)-3-(4-iodophenyl)-1-oxopropan-2-yl)carbamoyl)cyclohexyl)methanaminium trifluoroacetate following general procedure V-B. Preparatory HPLC purification was carried out using a method consisting of 8 min of 100% A after injection followed by a gradient from 0 to 100% B over 20 min. Fractions containing the desired compound were lyophilised to obtain Ik as white powder. (42 mg, 0.04 mmol, 53%) ¹H NMR (600 MHz, CD₃CN+10% D₂O) δ 7.60 (d, 2H), 6.98 (d, 2H), 4.43-4.34 (m, 1H), 4.20-4.11 (m, 1H), 4.11-4.00 (m, 1H), 3.71-3.39 (m, 8H), 3.31-2.98 (m, 16H), 2.98-2.85 (m, 2H), 2.78 (dd, J=13.5, 8.6 Hz, 1H), 2.39-2.28 (m, 2H), 2.10-1.97 (m, 2H), 1.87-1.74 (m, 1H), 1.73-1.61 (m, 4H), 1.60-1.44 (m, 3H), 1.41-1.27 (m, 1H), 1.27-1.09 (m, 4H), 0.92-0.74 (m, 3H).

¹³C NMR (151 MHz, CD₃CN+10% D₂O) δ 178.8, 176.8, 176.7, 176.1, 172.9, 162.1 (q, J=34.2), 159.5, 138.3, 138.1, 132.6, 118.8 (overlapped with CD₃CN signal, q, J=293 Hz), 92.5, 55.8, 55.3, 53.9, 53.3, 46.2, 45.3, 39.6, 37.8, 37.6, 32.0, 30.9, 30.3, 30.2, 29.6, 29.3, 29.1, 27.8, 23.3.

### (((S)-1-carboxy-5-((R)-3-(5-iodo-1H-indol-3-yl)-2-((1r,4R)-4-((2-(4,7,10-tris(carboxymethyl)-1,4,7,10-tetraazacyclododecan-1-yl)acetamido)methyl)cyclohexane-1-carboxamido)propanamido)pentyl)carbamoyl)-L-glutamic acid trifluoroacetic acid salt (Il)

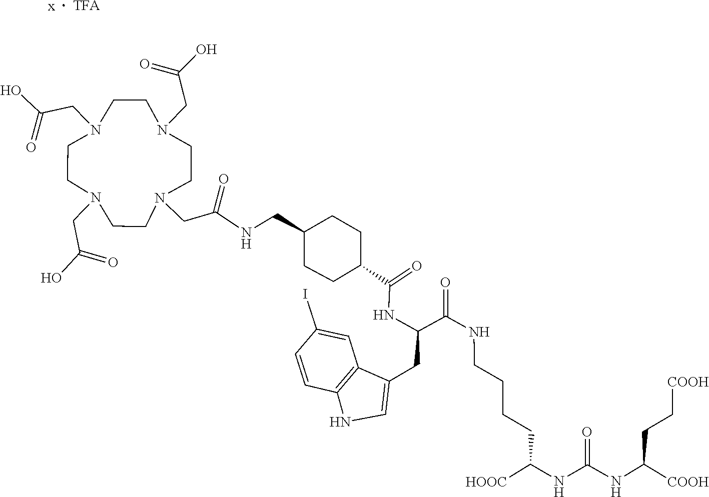

The title compound was obtained from ((1R,4r)-4-(((R)-1-(((S)-5-carboxy-5-(3-((S)-1,3-dicarboxypropyl)ureido)pentyl)amino)-3-(5-iodo-1H-indol-3-yl)-1-oxopropan-2-yl)carbamoyl)cyclohexyl)methanaminium trifluoroacetate following general procedure V-B. Preparatory HPLC purification was carried out using a method consisting of 8 min of 100% A after injection followed by a gradient from 0 to 100% B over 20 min. Fractions containing the desired compound were lyophilised to obtain Il as white powder. (14 mg, 0.01 mmol, 46%) ¹H NMR (600 MHz, CD₃CN+10% D₂O) δ 7.91 (s, 1H), 7.36 (dd, J=8.5, 1.7 Hz, 1H), 7.21 (d, J=8.5 Hz, 1H), 7.08 (d, J=8.3 Hz, 1H), 4.44 (t, J=7.1 Hz, 1H), 4.21 (dt, J=8.9, 4.4 Hz, 1H), 4.07 (dt, J=8.9, 4.9 Hz, 1H), 3.90-3.50 (m, 8H), 3.30-2.96 (m, 18H), 2.37 (td, J=7.0, 6.1, 2.9 Hz, 2H), 2.13-2.02 (m, 3H), 1.89-1.79 (m, 1H), 1.77-1.61 (m, 5H), 1.52 (dtd, J=14.0, 9.2, 5.3 Hz, 1H), 1.46-1.36 (m, 1H), 1.35-1.21 (m, 5H), 1.20-1.11 (m, 2H), 0.96-0.82 (m, 3H).

¹³C NMR (151 MHz, CD₃CN+10% D₂O) δ 178.3, 176.4, 176.2, 175.8, 173.2, 161.4 (q, J=34.7 Hz), 159.5, 136.3, 131.2, 130.5, 128.4, 126.0, 116.7 (overlapped with CD₃CN signal, q, J=292 Hz), 114.8, 110.3, 82.8, 56.0, 55.1, 53.9, 53.3, 46.4, 45.4, 39.6, 39.4, 37.8, 32.2, 32.0, 30.9, 30.4, 30.3, 29.5, 29.1, 29.0, 28.4, 28.0, 27.9, 23.4.

### Example 4: Synthesis of PSMA-Targeting Radiopharmaceutical Precursor

### di-tert-butyl (((S)-1-(tert-butoxy)-1-oxo-6-((S)-3-(4-(trimethylstannyl)phenyl)-2-((1r,4S)-4-((2-(4,7,10-tris(2-(tert-butoxy)-2-oxoethyl)-1,4,7,10-tetraazacyclododecan-1-yl)acetamido)methyl)cyclohexane-1-carboxamido)propanamido)hexan-2-yl)carbamoyl)-L-glutamate

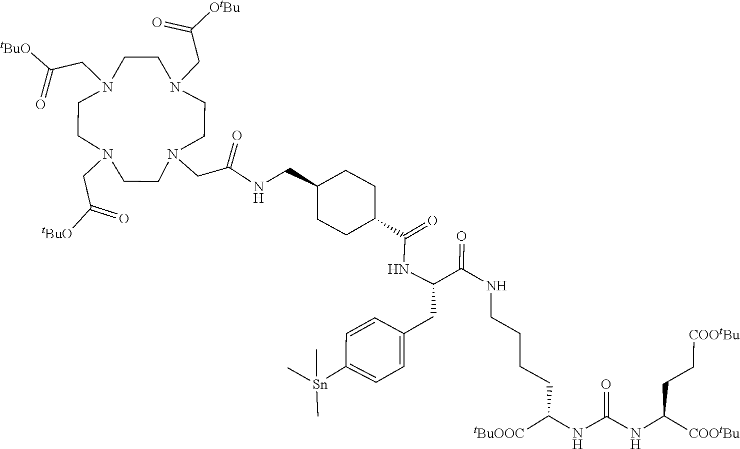

To an LuG-linker solution (1 eq.) in DCM, Et₃N (6 eq.), DOTA-mono-NHS-tris(tBu-ester) (1.2 eq.) were added and stirred overnight (˜16 hours). Afterwards the reaction mixture was evaporated under reduced pressure.

One microwave vial was charged with Pd(OAc)₂ (1.5 mg, 0.007 mmol) and the crude LuG-linker DOTA-functionalized mixture (7.7 mg, 0.26 mmol). The vial was sealed and purged. Then dry/degassed THF (400 uL) was added to the vial. In another MW vial, hexamethylditin (20.5 uL, 0.099 mmol) was added and the vial purged, thereafter dry/degassed THF was added (300 uL). The hexamehtylditin solution was then added to the Pd(OAc)₂ and meCgPPH mixture and the solution stirred for 5 minutes at room temperature.

Thereafter, fully tBu-protected 4 (48.0 mg, 0.033 mmol) which was previously weighed in a MW vial and dissolved in dry/degassed THF (300 uL) was added to the Pd/meCgPPH/Sn mixture. The vial was then placed on a MW system and heated to 70° C. for 30 min.

The mixture was dried under a stream of air and re-dissolved in 4 mL 60:40:0.1 (MeCN:H₂O:TFA). The mixture was filtered over a prep HPLC filter and injected into the prepHPLC system in a gradient of 60 to 100% B. which yielded the desired product as a white solid. (12.5 mg, 0.0084 mmol, 25%). MS (ESI) m/z: 747.47 [M+2H]²⁺

### Example 5: Radioastatination and Radiochemical Conversion (RCC)

Compound Ib was provided in a two-step labelling procedure:

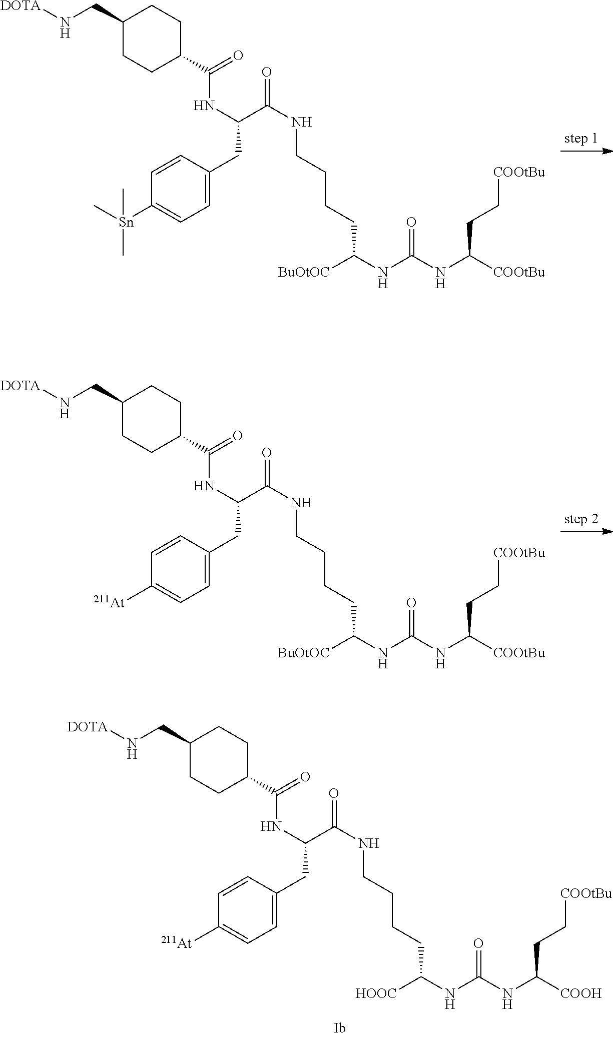

The stannane precursor was added to a solution of chloramine-T, methanol, ²¹¹At and acetic acid. The mixture was stirred for 30 minutes at room temperature (step 1), followed by drying under a nitrogen stream. The radioastatinated product was deprotected by addition of trifluoroacetic acid (TFA) and heating to 60° C. for 30 minutes (step 2). The radiochemical conversion (% RCC) for this procedure is shown in Table 1:

TABLE 1

| | % RCC | % RCC | % RCC | |
|---|---|---|---|---|
| Activity range | [radioTLC] | [radioTLC] | [TLC] | |
| | (Step 1) | (Step 2) | (overall) | n |
| 5-61 MBq | 81 ± 5 | 91 ± 6 | 71 ± 7 | 3 |

Thus, the PSMA analogue, provided in a two-step labeling procedure, resulted in a RCC of >60%, which is surprising, since a similar labelling procedure, reported in WO2019/157037 yielded a labelled PSMA analogue ([²¹¹At]VK-02-90) in 12.5% radiochemical yield (RCY, non-decay corrected), over two steps. No detailed data is provided for the method used in WO2019/157037 (one pot procedure, 3 modification), however an RCY of maximum 26% was reported.

RCC as stated above refers to radiochemical conversion, and is used as a measure of how much of the added activity that is converted to the desired product, as demonstrated by chromatography, typically radio-TLC or radio-HPLC. RCC measurements are made before a potential work-up or purification is carried out. In this way, RCC can be said to measure the efficiency of the chemical radiolabeling reaction. RCY refers to radiochemical yield, and is used as a measure of how much of the added activity ends up as the desired product in a purified form, typically with an associated radiochemical purity (RCP) that says how much of the activity in the purified product is present as the actual desired product. In this sense, RCY reflects both the efficiency of the labeling (RCC) and the efficiency of the work-up procedure, since product may be lost during purification. However, unless a very inefficient type of purification is used, RCY will be similar to RCC, with RCC being slightly higher. In WO2019/157037, purification was done by HPLC, a state-of-the-art, standard procedure. Accordingly, RCY and RCC would be expected to be similar, typically with a difference between the two of about 5-15%. In this sense, the difference between a reported RCY of 26% and an RCC of 71% is substantial and reflects a difference in the efficiency of the radiolabeling reactions themselves. It should be noted that efficient radiochemistry is crucial for commercial use as it limits loss of the radionuclide, makes purification easier, limits radioactive waste, and limits exposure of personnel to radiation.

### Example 6: 68Ga-Labeling

⁶⁸Ga [E_{β+,max}=1.9 MeV (88%), t_{1/2}=68 min] was eluted from a ⁶⁸Ge (t_{1/2}=271 d) generator (ITM AG, Munich, Germany), based on silica gel modified with dodecyl gallate, as [⁶⁸Ga]GaCl₃ in 0.1 M HCl and trapped in an SCX cartridge (HyperSep, ThermoFischer). The trapped [⁶⁸Ga]Ga³⁺ was eluted with 300 μL of a 5 M NaCl/HCl solution generally giving 500-600 MBq of activity and employed in further radiolabellings.

Radiolabelling of the DOTA-containing peptidomimetics was carried out as follows. 40 uL of [⁶⁸Ga]Ga³⁺ eluate (˜50-80 MBq) were mixed with 40 μL of 1.0 M HEPES (4-(2-hydroxyethyl)-1-piperazineethanesulfonic acid, pH 4). If needed, the pH of the solution was adjusted to 3.8-4.2 by addition of 10% NaOH_{(aq.)}. Thereafter, 5 μl (internalization experiment) or 2 μl (PET imaging) of a 1 mM solution of the DOTA-bearing peptide was added and the reaction mixture was heated to 95° C. for 15 min (internalization experiment) or 5 min (PET imaging), respectively. Radiolabelling efficiency was determined by RP-HPLC (5-95% B in 5 minutes—Chromolith RP-18e 100×4.6) and was deemed to be >98% for all the radiolabelled compounds.

### Example 7: In Vitro Test of Binding Affinity and Internalisation

Compounds Ii, Ij, Ik, Il, Im and In was provided using the same method as disclosed in Example 3 and in vitro test of the binding affinity and internalisation of the compounds were examined using the method as described in Benesova et al, 2016⁹. The iodine isotope used was ¹²⁷I.

For internalisation determinations, a day before the experiment, PSMA(+) LNCaP cells were seeded in a poly-L-lysine coated 24-well plate (10⁵ cells per well) and maintained at 37° C. in an atmosphere of 5% CO₂ under supplemented RMPI medium (10% Fetal Calf Serum, 1% sodium pyruvate, 1% FIS). Cells were incubated with 250 μL of radiolabelled compound diluted in RPMI medium (final concentration of [⁶⁸Ga]-peptide: 30 nM) and 500 μM of PMPA (2-phosphonomethyl-pentanedioic acid) for the blocked series, for 45 min at 37° C. Cellular uptake was interrupted by washing the cells with ice-cold PBS (3×1 mL). Surface bound radioactivity was removed by incubating twice with 0.5 mL glycine (50 mM, pH=2.8) for 5 min. Thereafter, cells were washed with PBS (1 mL) and lysed employing NaOH (0.3 M) during 10 min. Lysates and surface-bound activity were collected and measured in a gamma-counter (Perkin Elmer 2480, Wizard, Gamma Counter). The cell uptake was calculated as percent of the initially added radioactivity bound to 105 cells [% ID/105 cells].

For internalisation studies androgen-sensitive human prostate adenocarcinoma cells (LNCaP) highly overexpressing PSMA were incubated with the ⁶⁸Ga-labeled compounds resulting in specific cell surface binding of all tested compounds (Table 2).

TABLE 2
Internalisation data*.

| | Specifically cell | Specifically |
|---|---|---|
| | surface bound | internalised |
| Compound | [% IA/10⁵ cells]^{†} | [% IA/10⁵ cells]^{†} |
| PSMA-617 | 1.5 ± 0.4 | 0.6 ± 0.2 |
| II | 1.1 ± 0.4 | 0.7 ± 0.1 |
| U | 2.3 ± 0.2 | 0.8 ± 0.3 |
| IK | 0.7 ± 0.1 | 0.6 ± 0.3 |
| IL | 1.6 ± 0.4 | 0.6 ± 0.2 |
| IN | 0.4 ± 0.1 | 0.2 ± 0.1 |
| IM | 1.6 ± 0.5 | 0.9 ± 0.5 |

*Data are expressed as mean ± SD (n=3),
^{†68}Ga-labeled compounds. Specific cell uptake was determined by blockage using 500 μM 2-PMPA. Values are expressed as % of applied radioactivity (IA) bound to 10⁵ cells.

The results are shown in Table 2. All compounds revealed comparable internalisation properties as PSMA-617, except IN showing a reduced internalised fraction. Especially compound Ij and Im displayed a specific internalization higher than or comparable to PSMA-617, which was surprising, as modifying this amino acid residue in the linker region of PSMA inhibitors can have significant effects on binding and internalisation, as is reported in Benesova et al., 2016⁹. Internalisation is the key predictor for successful PSMA therapy.

### Example 8: In Vivo Evaluation of the PSMA Targeting Radioligands

Compounds Ii, Ij, Ik, Il and Im which showed internalisation comparable to PSMA-617 as shown in Example 7 were selected for in vivo evaluation in mice.

For the experimental tumor models 1×10⁷ cells of LNCaP (in 50% Matrigel; Becton Dickinson) were subcutaneously inoculated into the right flank of 7- to 8-week-old male BALB/c nu/nu mice (Janvier). For imaging studies, mice were anesthetized (2% isoflurane) and 0.5 nmol of the ⁶⁸Ga-labeled compound in 0.9% NaCl (pH 7) were injected into the tail vein. PET imaging was performed with μPET/MRI scanner (BioSpec 3T, Bruker) with a dynamic scan for 60 min. The images were iteratively reconstructed (MLEM 0.5 algorithm, 12 iterations) and were converted to SUV images. Quantification was done using a ROI (region of interest) technique and data in expressed in time activity curves as SUV_{body weight}. All animal experiments complied with the current laws of the Federal Republic of Germany.

### PET Imaging: Tumor Uptake and Pharmacokinetic Profile

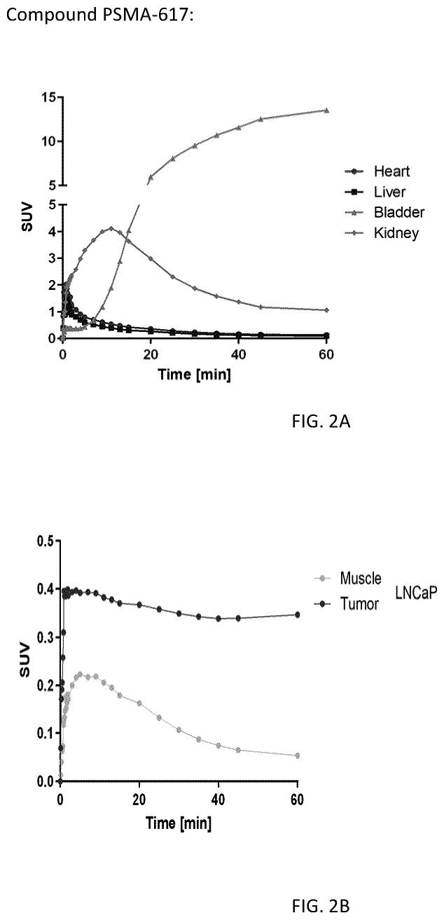

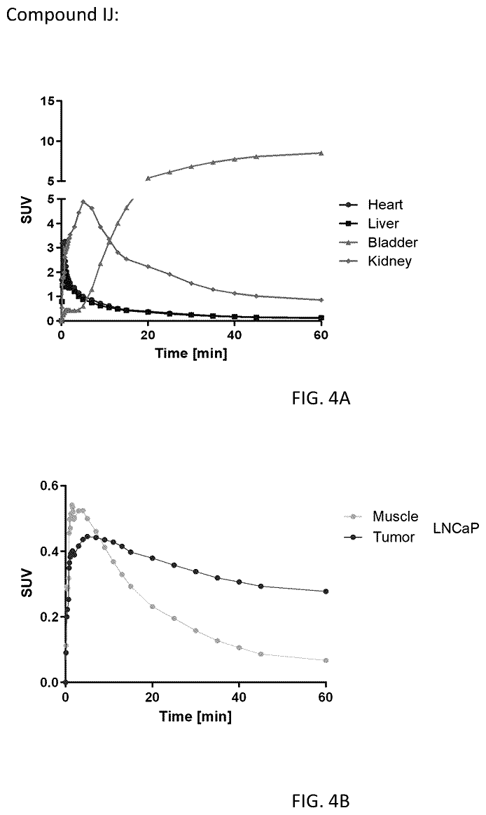

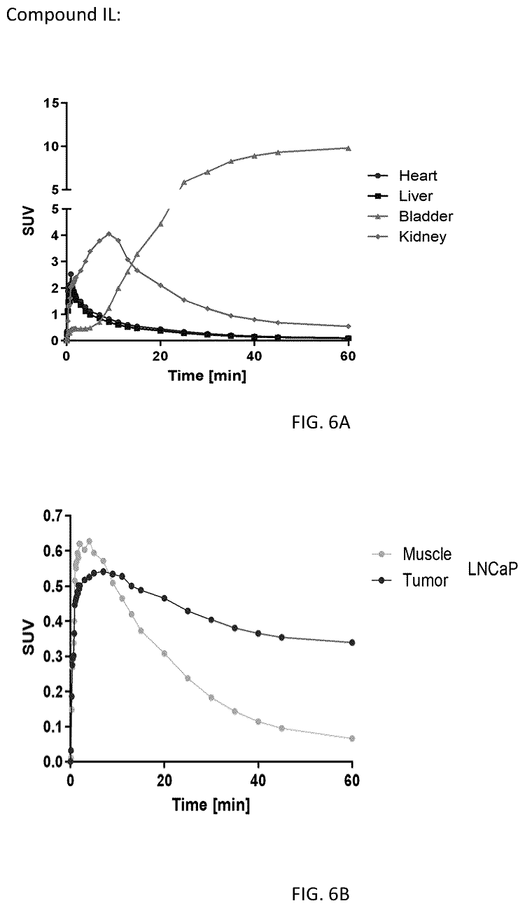

FIGS. 2 to 7 show the pharmacokinetic study with small-animal PET imaging. Time activity curves for non-target organs and tumor after injection of 0.5 nmol ⁶⁸Ga-labeled compounds in LNCaP-tumor-bearing athymic nude mice (right trunk) up to 60 min p.i. SUV=standardized uptake value.

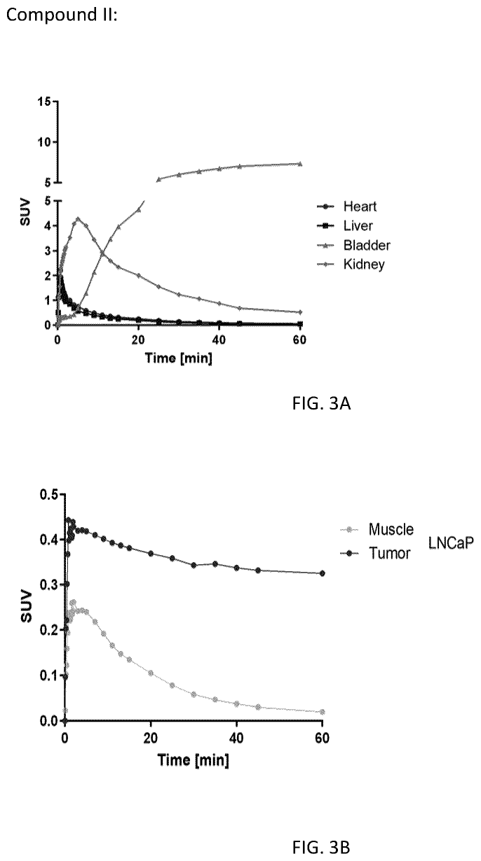

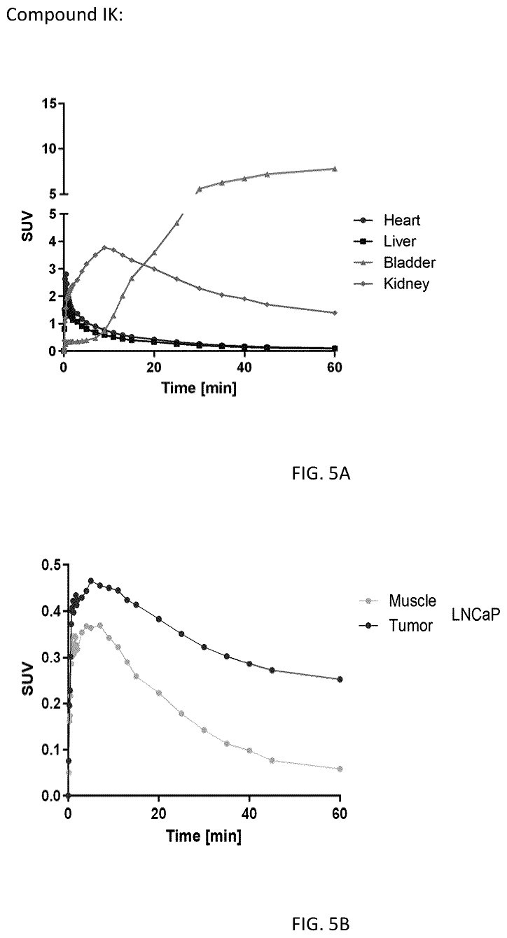

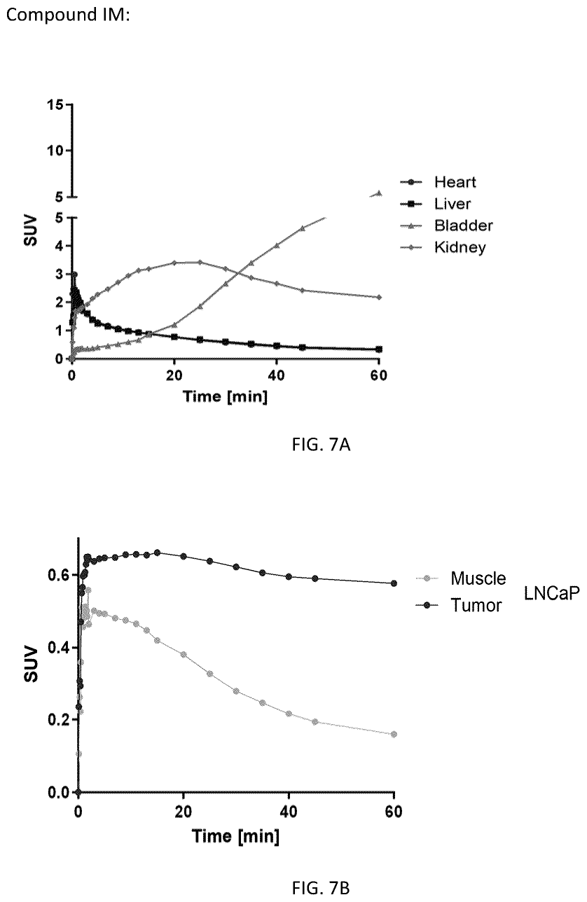

The pharmacokinetic properties and tumor targeting properties of the modified compounds were found to be comparable or superior to the parental reference PSMA-617 (see FIGS. 2-7). For example and surprisingly, the tumor-to-muscle ratio for Ii increased at later time points compared to PSMA-617 (FIG. 3B). Im (FIG. 7B) showed a higher tumor uptake compared to PSMA-617 (FIG. 2B), whereas a more reversible tumor accumulation could be seen for IK (FIG. 5B). Moreover, the total uptake in the investigated organs and tumor, as well as the excretion profile, indicate the suitability of the new compounds as radiopharmaceuticals—for example as 211At labeled PSMA-inhibitors.

### REFERENCES

(1) Bray, F.; Ferlay, J.; Soerjomataram, I.; Siegel, R. L.; Torre, L. A.; Jemal, A. Global Cancer Statistics 2018: GLOBOCAN Estimates of Incidence and Mortality Worldwide for 36 Cancers in 185 Countries. CA. Cancer J. Clin. 2018, 68 (6), 394-424.
(2) Antonarakis, E. S.; Feng, Z.; Trock, B. J.; Humphreys, E. B.; Carducci, M. A.; Partin, A. W.; Walsh, P. C.; Eisenberger, M. A. The Natural History of Metastatic Progression in Men with Prostate-Specific Antigen Recurrence after Radical Prostatectomy: Long-Term Follow-Up. BJU Int. 2012, 109 (1), 32-39.
(3) De Bono, J. S.; Oudard, S.; Ozguroglu, M.; Hansen, S.; MacHiels, J. P.; Kocak, I.; Gravis, G.; Bodrogi, I.; MacKenzie, M. J.; Shen, L.; et al. Prednisone plus Cabazitaxel or Mitoxantrone for Metastatic Castration-Resistant Prostate Cancer Progressing after Docetaxel Treatment: A Randomised Open-Label Trial. Lancet 2010, 376 (9747), 1147-1154.
(4) Israeli, R. S.; Powell, C. T.; Corr, J. G.; Fair, W. R.; Heston, W. D. W. Expression of the Prostate-Specific Membrane Antigen. Cancer Res. 1994, 54 (7), 1807-1811.
(5) Benesova, M.; Schafer, M.; Bauder-Wüst, U.; Afshar-Oromieh, A.; Kratochwil, C.; Mier, W.; Haberkorn, U.; Kopka, K.; Eder, M. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56 (6), 914-920.
(6) Pomper, M. G.; Mease, R. C.; Chen, Y.; Ray, S.; Zalutsky, M.; Vaidyanathan, G. PSMA TARGETED RADIOHALOGENATED UREAS FOR CANCER RADIOTHERAPY—WO2017070482A2, 2016.
(7) Pomper, M. G.; Mease, R. C.; Kumar, V.; Ray, S.; Zalutsky, M.; Vaidyanathan, G. PSMA TARGETED RADIOHALOGENATED UREA-POLYAMINOCARBOXYLATES FOR CANCER RADIOTHERAPY WO2019157037A1, 2019.
(8) Lowe, P. T.; Dall'Angelo, S.; Fleming, I. N.; Piras, M.; Zanda, M.; O'Hagan, D. Enzymatic Radiosynthesis of a 18 F-Glu-Ureido-Lys Ligand for the Prostate-Specific Membrane Antigen (PSMA). Org. Biomol. Chem. 2019, 17 (6), 1480-1486.
(9) Benešová, M.; Bauder-Wüst, U.; Schäfer, M.; Klika, K. D.; Mier, W.; Haberkorn, U.; Kopka, K.; Eder, M. Linker Modification Strategies to Control the Prostate-Specific Membrane Antigen (PSMA)-Targeting and Pharmacokinetic Properties of DOTA-Conjugated PSMA Inhibitors. J. Med. Chem. 2016, 59 (5), 1761-1775.
(10) Feuerecker B., Tauber, R., Knorr, K. Heck, M., Beheshti, A., Seidl, C., Bruchertseifer, F., Pickhard, A., Gafita, A., Kratochwil, C., Retz, M., Gschwend, J. E., Weber, W. A, D'Alessandria, C., Morgenstern, A., Eiber, M. Activity and Adverse Events of Actinium-225-PSMA-617 in Advanced Metastatic Castration-resistant Prostate Cancer After Failure of Lutetium-177-PSMA. European Urology, 2021, 79 (3), 343-350.
(11) Iley, J., Tolando, R., Constantino, L. Chemical and microsomal oxidation of tertiary amides: regio- and stereoselective aspects. J. Chem. Soc., Perkin Trans. 2, 2001, 1299-1305
(12) Syrén, P. O. Enzymatic Hydrolysis of Tertiary Amide Bonds by anti Nucleophilic Attack and Protonation. J. Org. Chem. 2018, 83, 21, 13543-13548

1. A PSMA targeting ligand of formula (I) 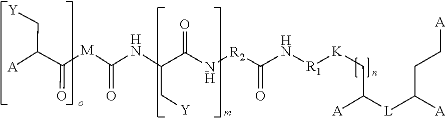
wherein:
A is independently carboxylic acid, sulphonic acid, phosphonic acid, tetrazole or isoxazole;
L is selected from the group consisting of urea, thiourea, —NH—(C═O)—O—, —O—(C═O)—NH— or —CH₂—(C═O)—CH₂—;
K is selected from the group consisting of —(C═O)—NH—, —CH₂—NH—(C═O)— or 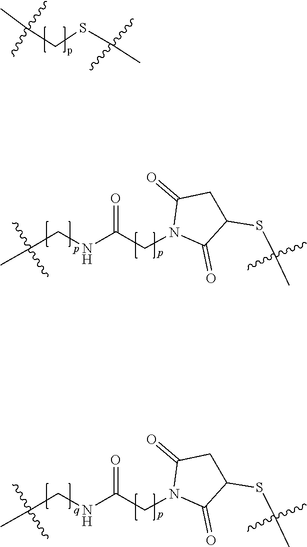
wherein
p is independently an integer selected from the group consisting of 1, 2, 3, 4, 5 and 6;
q is an integer selected from the group consisting of 0, 1, 2, 3, 4, 5 and 6;
Y is selected from the group consisting of: 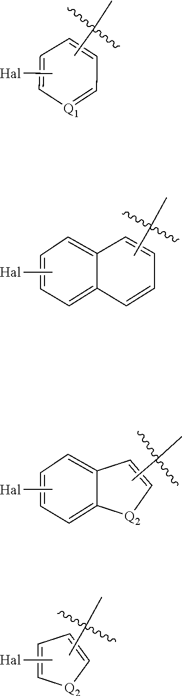
wherein
Q₁ is —C—R₃ or N, wherein R₃ is H or C₁-C₅ alkyl;
Q₂ is O, S or NH;
Hal is a nuclide or a radionuclide of the halogen group selected from the group consisting of isotopes and radioisotopes of fluorine, iodine, bromine or astatine;
M is a chelating agent, that can comprise a metal;
n is an integer selected from the group consisting of 1, 2, 3, 4, 5 and 6;
m is an integer selected from the group consisting of 0 and 1;
o is an integer selected from the group consisting of 0 and 1;
R₁ is —CH—CH₂—Z or —CH—CH₂—Y;
wherein Z is selected from the group consisting of: 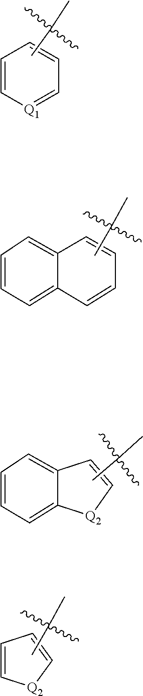
and Y is selected from the group consisting of: 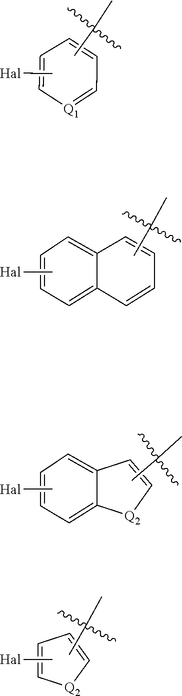
wherein
Q₁ is —C—R₃ or N, wherein R₃ is H or C₁-C₅ alkyl;
Q₂ is O, S or NH;
Hal is a nuclide or radionuclide of the halogen group selected from the group consisting of isotopes and radioisotopes of fluorine, iodine, bromine or astatine;
R₂ is —CH—CH₂—Y or —CH₂—X—;
wherein X is an aromatic monocyclic or polycyclic ring system having 6 to 14 carbon atoms, cyclopropyl, cyclobutyl, cyclopentyl, cyclohexyl, cycloheptyl or cyclooctyl;
and Y is selected from the group consisting of: 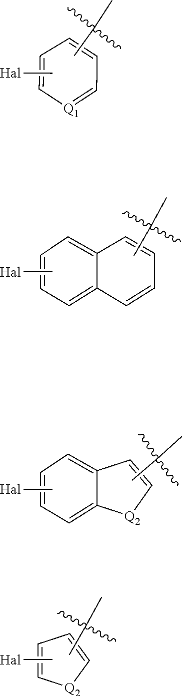
wherein
Q₁ is —C—R₃ or N, wherein R₃ is H or C₁-C₅ alkyl;
Q₂ is O, S or NH;
Hal is a nuclide or radionuclide of the halogen group selected from the group consisting of isotopes and radioisotopes of fluorine, iodine, bromine or astatine;
and wherein formula (I) comprises at least one isotope or radioisotope selected from fluorine, iodine, bromine or astatine;
and pharmaceutically acceptable salts thereof.

2. A PSMA targeting ligand according to claim 1, having the general formula (Ia): 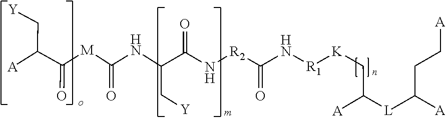
wherein:
A is independently carboxylic acid, sulphonic acid, phosphonic acid, tetrazole or isoxazole;
n is an integer selected from the group consisting of 1, 2, 3 and 4;
m is an integer selected from the group consisting of 0 and 1;
o is an integer selected from the group consisting of 0 and 1;
is is selected from the group consisting of: 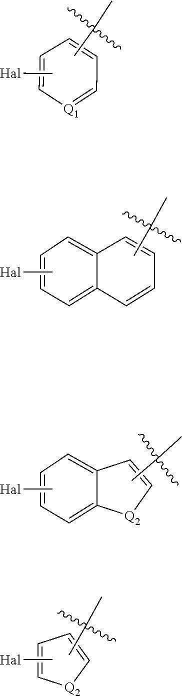
wherein
Q₁ is —C—R₃ or N, wherein R₃ is H or C₁-C₅ alkyl;
Q₂ is O, S or NH;
Hal is a nuclide or radionuclide of the halogen group selected from the group consisting of isotopes and radioisotopes of fluorine, iodine, bromine or astatine;
M is a chelating agent, that can comprise a metal;
R₁ is —CH—CH₂—Z or —CH—CH₂—Y;
wherein Z is selected from the group consisting of: 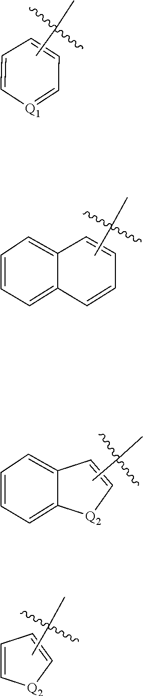
and Y is selected from the group consisting of: 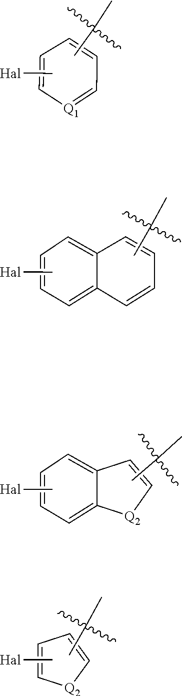
wherein
Q₁ is —C—R³ or N, wherein R³ is H or C₁-C₅ alkyl;
Q₂ is O, S or NH;
Hal is a nuclide or radionuclide of the halogen group selected from the group consisting of isotopes and radioisotopes of fluorine, iodine, bromine or astatine;
R₂ is —CH—CH₂—Y or —CH₂—X—;
wherein X is an aromatic monocyclic or polycyclic ring system having 6 to 14 carbon atoms, cyclopropyl, cyclobutyl, cyclopentyl, cyclohexyl, cycloheptyl or cyclooctyl;
and Y is selected from the group consisting of: 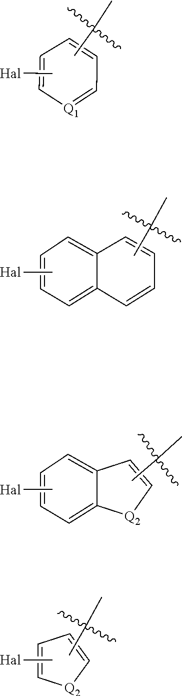
wherein
Q₁ is —C—R³ or N, wherein R³ is H or C₁-C₅ alkyl;
Q₂ is O, S or NH;
Hal is a nuclide or radionuclide of the halogen group selected from the group consisting of isotopes and radioisotopes of fluorine, iodine, bromine or astatine;
and wherein formula (I) comprises at least one isotope or radioisotope selected from fluorine, iodine, bromine or astatine;
and pharmaceutically acceptable salts thereof.

3. The PSMA targeting ligand according to claim 1,
wherein M is selected from the group consisting of:
1,4,7,10-tetraazacyclododecane-N,N′,N′,N″-tetraacetic acid (DOTA),
N,N′-bis(2-hydroxy-5-(carboxyethyl)benzyl)ethylenediamine N,N′-diacetic acid (HBED-CC),
14,7-triazacyclononane-1,4,7-triacetic acid (NOTA),
2-(4,7-bis(carboxymethyl)-1,4,7-triazonan-1-yl)pentanedioic acid (NODAGA),
2-(4,7,10-tris(carboxymethyl)-1,4,7,10-tetraazacyclododecan-1-yl)pentanedioic acid DOTAGA),
4,7-triazacyclononane phosphinic acid (TRAP), 14,7-triazacyclononane methyl(2-carboxyethyl)phosphinic acid-4,7-bis(methyl(2-hydroxymethyl)phosphinic acid (NOPO),
3,6,9,15-tetraazabicyclo9.3.1.pentadeca-1(15),11,13-triene-3,6,9-triacetic acid (PCTA),
N′-(5-acetyl (hydroxy)aminopentyl-N-(5-(4-(5-aminopentyl)(hydroxy)amino-4-oxobutanoyl)amino)pentyl-N-hydroxysuccinamide (DFO),
diethylenetriaminepentaacetic acid (DTPA),
trans-cyclohexyl-diethylenetriaminepentaacetic acid (CHX-DTPA),
1-oxa-4,7,10-triazacyclododecane-4,7,10-triacetic acid (OXO-Do3A),
p-isothiocyanatobenzyl-DTPA (SCN-BZ-DTPA),
1-(p-isothiocyanatobenzyl)-3-methyl-DTPA (1B3M),
2-(p-isothiocyanatobenzyl)-4-methyl-DTPA (1M3B), and
1-(2)-methyl-4-isocyanatobenzyl-DTPA (MX-DTPA);
and pharmaceutically acceptable salts thereof.

4. The PSMA targeting ligand according to claim 3, wherein M comprises a metal selected from the group consisting of Y, Lu, Tc, Zr, In, Sm, Re, Cu, Pb, Ac, Bi, Al, Ga, Ho and Sc.

5. The PSMA targeting ligand according to claim 1, wherein R₁ is —CH—CH₂—Y and Hal is selected from the group consisting of ¹⁸F, ¹⁹F, ¹²⁵I, ¹²³I, ¹³¹I, ¹²⁴I, ¹²⁷I, ²¹¹At, ⁷⁷Br, ⁸⁰Br, ⁷⁹Br, and ⁸¹Br

6. The PSMA targeting ligand according to claim 1, selected from the group consisting of: 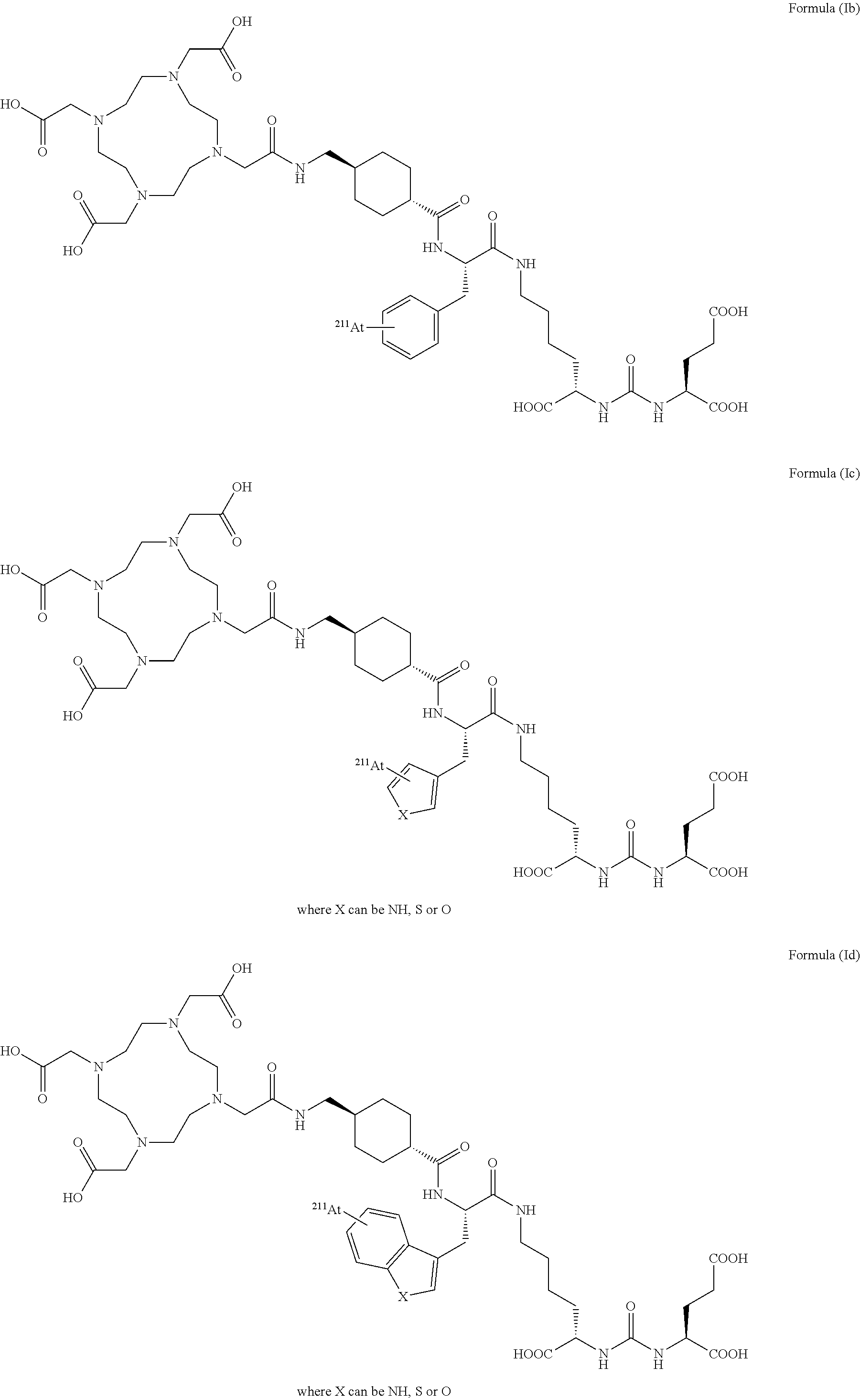 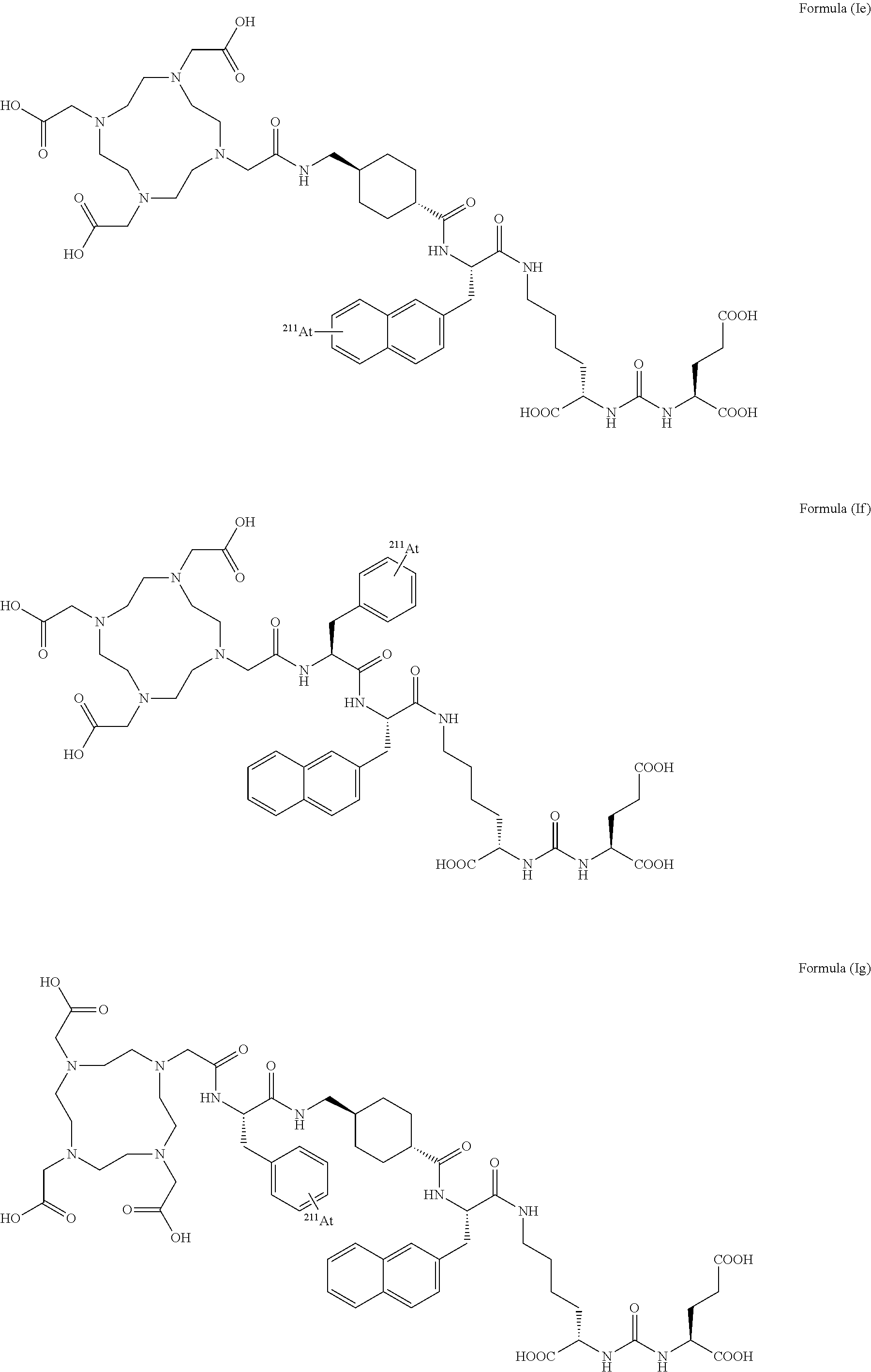 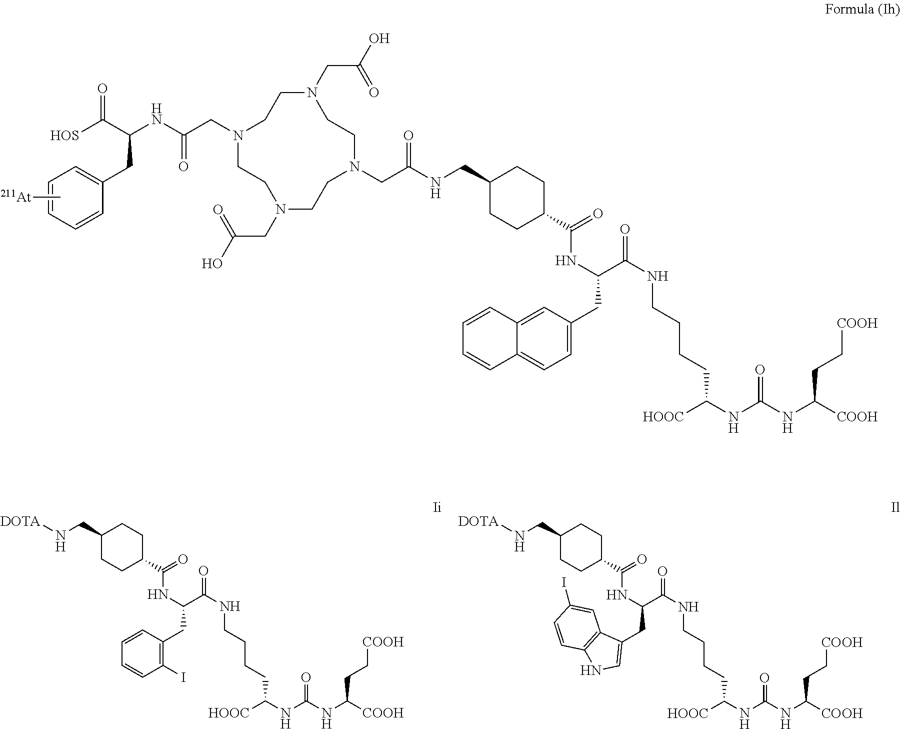 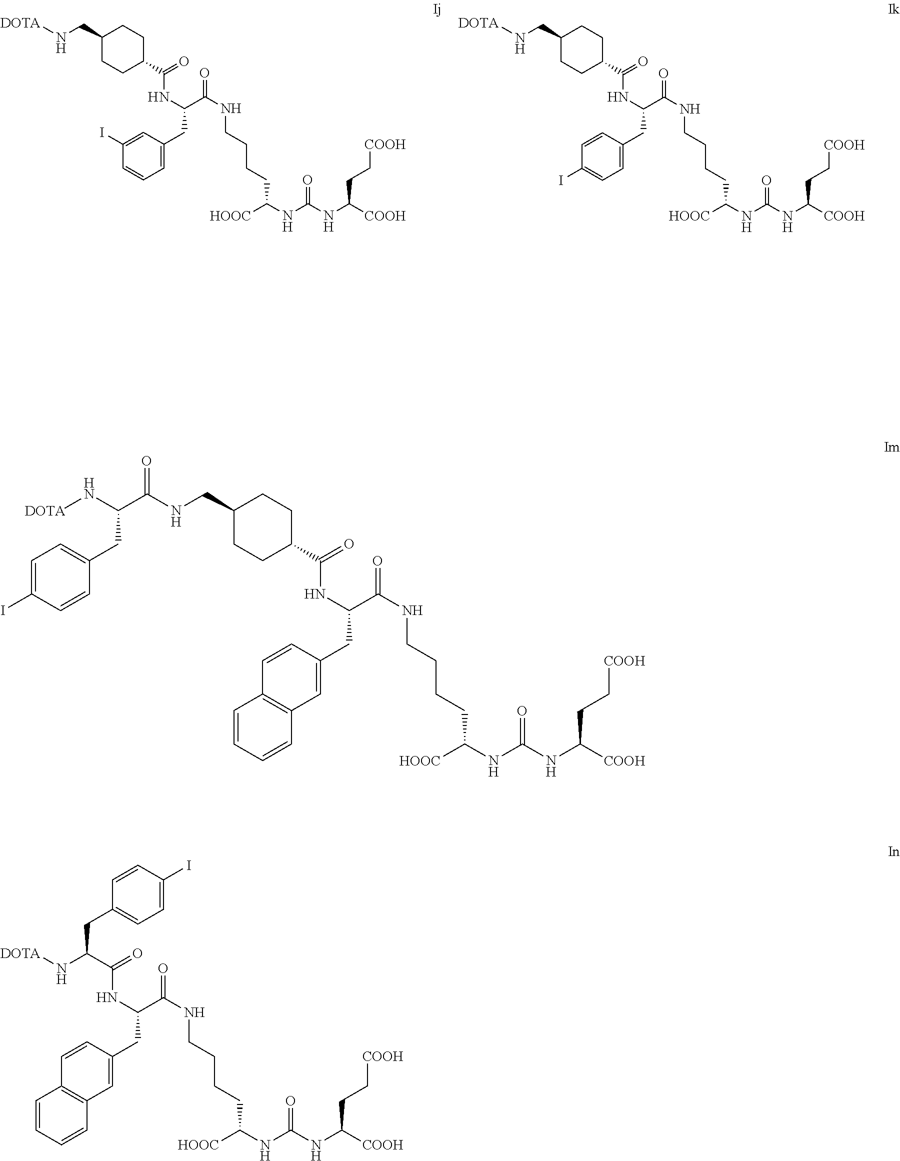
wherein in compound Ii, Ij, Ik, Il, Im and In “I” is an isotope or radioisotope of iodine.

7. A precursor compound of the PSMA targeting ligand according to claim 1 selected from the group comprising the below formulas (II) to (VIII) and the same precursor compounds wherein the Me)₃Sn group is replaced with a silyl, boron, iodonium or diazonium group:

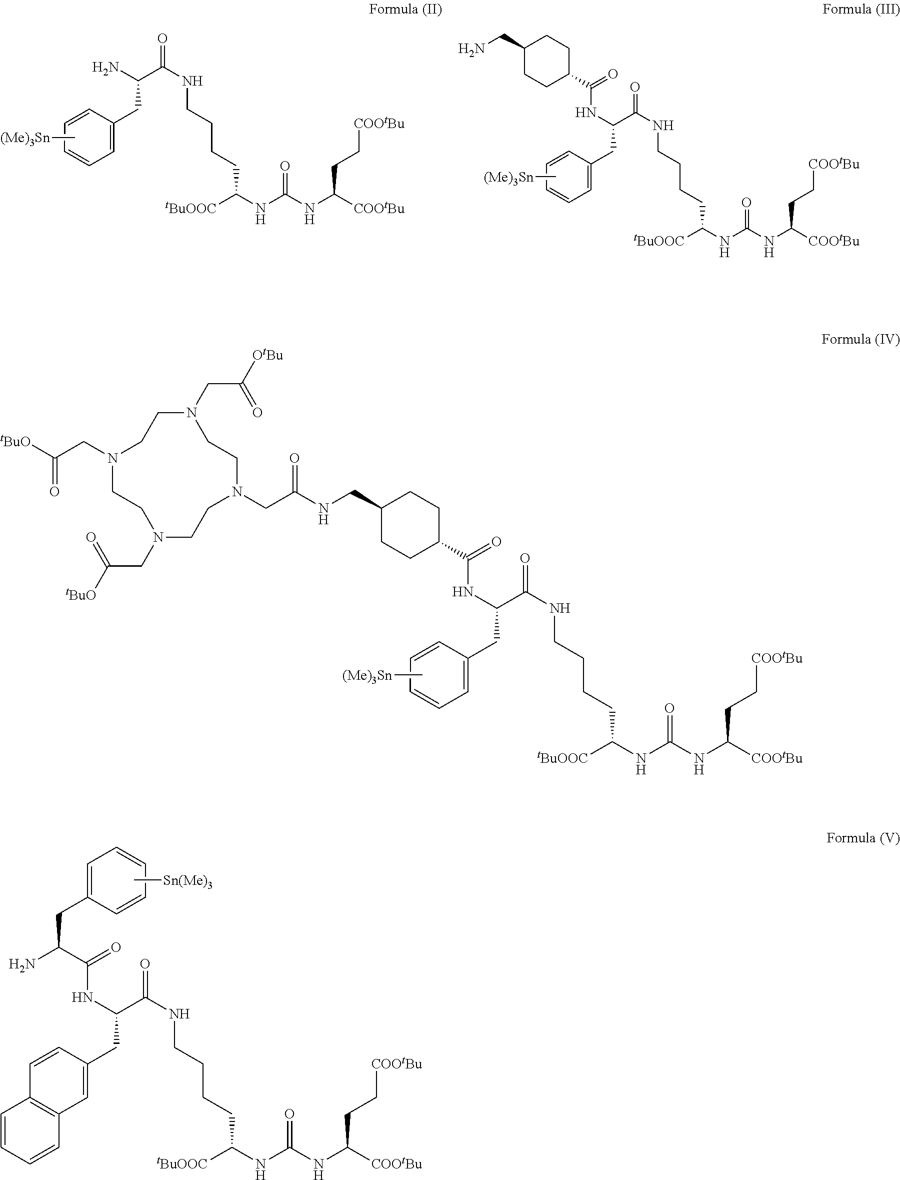

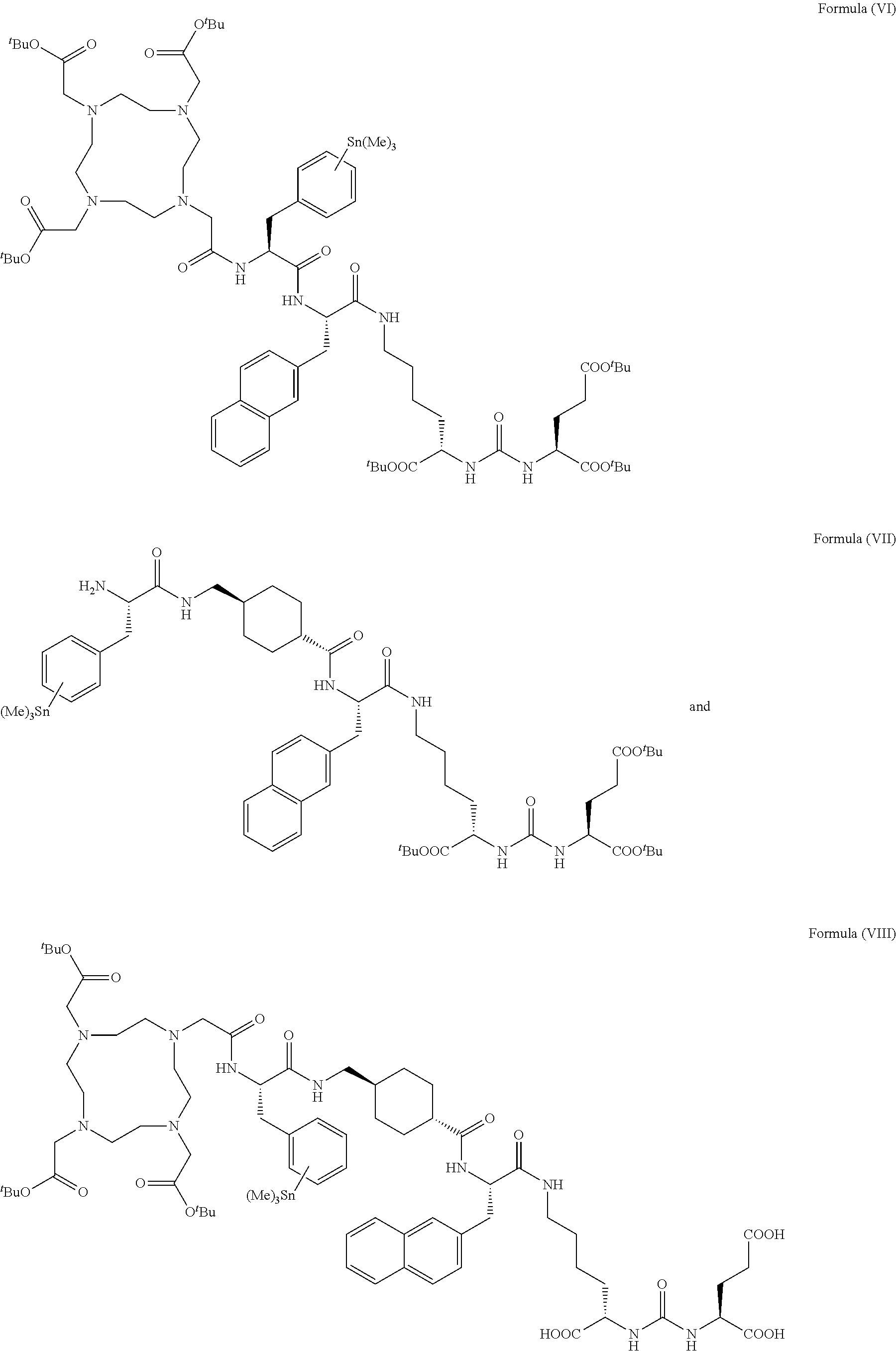

8. A method for providing the PSMA targeting ligand according to claim 1 comprising
Synthesis of a PSMA binding motif
Coupling of linkers to the PSMA binding motif, wherein one or more of the precursors of formula (II), (III), (V) and (VII) according to claim 7 is provided
Coupling of the PSMA binding motif-linker to a chelator wherein one or more of the precursors of Formula (IV), (VI) and (VIII) according to claim 7 is provided
Labeling the PSMA binding motif-linker-chelator with a halogen nuclide or radionuclide.

9. The method according to claim 8, wherein the PSMA binding motif is Lys-urea-Glu.

10. The method according to claim 9 wherein the halogen nuclide is selected from the group consisting of ¹⁸F, ¹⁹F, ¹²⁵I, ¹²³I, ¹³¹I, ¹²⁴I, ¹²⁷I, ²¹¹At, ⁷⁷Br, ⁷⁹Br, ⁸⁰Br, and ⁸¹Br.

11. The PSMA targeting ligands of formula (I) according to claim 1 wherein the halogen is selected from the group consisting of ²¹¹At, ¹²⁵I, ¹²³I, ⁷⁷Br, and ⁸⁰Br for use in radiotherapy.

12. The PSMA targeting ligands of formula (I) according to claim 1 wherein the halogen is ²¹¹At for use in the treatment of cancer, in particular prostate cancer.

13. The PSMA targeting ligands of formula (I) according to claim 1 wherein the halogen is selected from the group consisting of ¹²⁵I, ¹²³I, ¹³¹I, ¹²⁴I, ⁷⁷Br and ⁸⁰Br for use as a theranostic agent.

14. The use of PSMA targeting ligands of formula (I) according to claim 1 wherein the halogen is selected from the group consisting of ¹²⁵I, ¹²³I, ¹³¹I, ¹²⁴I, ⁷⁷Br and ⁸⁰Br as an imaging agent.

15. The use of PSMA targeting ligands of formula (I) according to claim 1 wherein the halogen is selected from the group consisting of ¹⁹F, ¹²⁷I, ⁷⁹Br, and ⁸¹Br as test-compounds.
